(19) United States
(12) Patent Application Publication
(10) Pub. No.: US 2024/0067621 A1
(43) Pub. Date: Feb. 29, 2024

(54) POLYFLUORINATED CANNABINOID AND CANNABINOID-LIKE COMPOUNDS AND METHODS OF SYNTHESIS AND USE THEREOF

(71) Applicants: Jimmie D. WEAVER, Stillwater, OK (US); Jon DAY, Stillwater, OK (US); Sascha GROTJAHN, Regensburg (DE); Sameera SENAWEERA, Minneapolis, MN (US); Burkhard KOENIG, Regensburg (DE); The Board of Regents For The Oklahoma Agricultural And Mechanical Colleges, Stillwater, OK (US)

(72) Inventors: Jimmie D. WEAVER, Stillwater, OK (US); Jon DAY, Stillwater, OK (US); Sascha GROTJAHN, Regensburg (DE); Sameera SENAWEERA, Minneapolis, MN (US); Burkhard KOENIG, Regensburg (DE)

(21) Appl. No.: 18/547,251

(22) PCT Filed: Feb. 22, 2022
(86) PCT No.: PCT/US22/17215

§ 371 Date: Aug. 21, 2023

Related U.S. Application Data
(60) Provisional application No. 63/152,674, filed on Feb. 23, 2021.

(51) Int. Cl. C07D 311/80; C07C 33/46; C07C 69/78

(52) U.S. Cl. CPC C07D 311/80; C07C 33/46; C07C 69/78

(57) ABSTRACT

Polyfluorinated compounds are disclosed, including polyfluorinated cannabinoid and cannabinoid-like compounds. Also disclosed are methods of producing the polyfluorinated compounds.

### CROSS REFERENCE TO RELATED APPLICATIONS/INCORPORATION BY REFERENCE STATEMENT

This application claims benefit under 35 USC § 119(e) of U.S. Provisional Application No. 63/152,674, filed Feb. 23, 2021. The entire contents of the above-referenced application(s) are hereby expressly incorporated herein by reference.

### STATEMENT REGARDING FEDERALLY SPONSORED RESEARCH OR DEVELOPMENT

This invention was made with Government support under Grant No. 1R01GM115697-01 awarded by the National Institutes of Health. The Government has certain rights in the invention.

### BACKGROUND

Fluorine has a greater ability than any other element to dramatically alter—and often enhancing—the properties of a molecule without dramatically altering its shape or function. Perhaps due to the fact that there are few natural products that contain fluorine, which are usually toxic derivatives of fluoroacetate, fluorination was historically overlooked as a legitimate direction for investigation. In 1953, however, a fluorinated analog to hydrocortisone, fludrocortisone, was introduced to the literature. It was found to have a tenfold increase in glucocorticoid activity and up to 800 times the mineralocorticoid activity when comparted to the non-fluorinated hormone, cortisol. As time has passed, fluorination has assumed a lead role in discovery chemistry because of its ability to improve a host of properties in a multitude of applications. To further emphasize, an appreciation for the importance of fluorination in pharmaceuticals can be gained from examination of the drugs approved per annum by the US FDA, of which typically 30% contain a C—F bond. In 2018, of the small molecule entities that were approved by the US FDA, 17 contained a C—F bond, 12 were fluoroarenes, and 3 were polyfluorinated arenes (baloxavir marboxil, larotrectinib, and bictegravir), indicating that arene polyfluorination specifically is becoming an increasingly important synthetic objective.

Despite the importance of fluorinated molecules in the pharmacopeia, linear syntheses to include fluorine are difficult. In contrast to more modern procedures, methods for sequential fluorine installation such as the Balz-Schiemann and halex reactions exist, but are difficult and are not typically functional group tolerant. Recent developments have increased the number of functional groups that can be transformed into fluorine. However, while these methods are useful for monofluorination, these methods contribute little in the exploration of multifluorinated molecules, because they would require a library of multi-site prefunctionalized non-fluorinated starting materials in order to perform a comprehensive structure activity relationship study (SAR), and these starting materials are generally not available.

Therefore, there is a need in the art for new and improved polyfluorinated compositions, as well as methods of producing and using same. It is to such new and improved compositions and methods that the present disclosure is directed.

### BRIEF DESCRIPTION OF THE DRAWINGS

FIG. 1 graphically compares a dearomatization reaction in accordance with the present disclosure (Scheme 1f) to various dearomatization reactions of the prior art (Schemes 1a-1e).

FIG. 2 describes the optimization of reaction conditions for production of polyfluorinated dicyclic compounds in accordance with the present disclosure.

FIG. 3 photographically shows how the presence of water significantly enhanced the outcome of the production reaction. The image demonstrates a darkening of each production reaction as an inverse function of equivalents of water added. Darkening of the reaction mixture could be avoided by the addition of water.

FIG. 4 includes one non-limiting embodiment of a method of producing polyfluorinated dicyclic compounds in accordance with the present disclosure (upper panel), as well as various non-limiting embodiments of chemical structures of compounds constructed in accordance with the present disclosure (lower panel).

FIG. 5 schematically depicts one non-limiting embodiment of a polyfluorinated dicyclic compound (11) constructed in accordance with the present disclosure and formed from Boc-protected methyl paraben as one of the coupling partners, as well as cleavage thereof with TFA (Scheme 2).

FIG. 6 schematically depicts deuteration (Scheme 3a) and kinetic isotope effect (KIE; Scheme 3b) reactions utilized in certain non-limiting embodiments of the polyfluorinated dicyclic compound production methods of the present disclosure.

FIG. 7 schematically depicts one non-limiting embodiment of a potential mechanistic understanding of the production methods of the present disclosure based upon the experiments and observations discussed in Example 1 (Scheme 4).

FIG. 8 schematically depicts certain non-limiting embodiments of THC fluoroanalogs constructed in accordance with the present disclosure (Scheme 5a), compared to THC (upper right) and other prior art cannabinoid fluoroanalogs (Scheme 5b).

FIG. 9 schematically depicts a deoxygenation reaction utilized with polyfluorinated dicyclic compounds constructed in accordance with the present disclosure (Scheme 6). Reagents and conditions: (a) ZnI₂ (1.5 equiv.), NaBH₃CN (7.5 equiv.), DCE 80° C., 97%; (b) bis(trifluoromethyl)benzoyl chloride (1.1 equiv.), 80° C., 89%; (c) DIPEA (2.0 equiv.), (Ir(dtbbpy)(dtbppy)₂]PF₆ (1.5 mol %), 45° C., 455 nm irradiation, 86%.

FIG. 10 schematically depicts a reduction of the Michael System for use with the polyfluorinated dicyclic compounds of the present disclosure (Scheme 7).

FIG. 11 schematically depicts an intramolecular cyclization reaction for use with the polyfluorinated dicyclic compounds of the present disclosure (Scheme 8).

FIG. 12 includes various non-limiting embodiments of chemical structures of polyfluorinated dicyclic compounds constructed in accordance with the present disclosure.

FIG. 13 includes various non-limiting embodiments of chemical structures of compounds constructed in accordance with the present disclosure.

FIG. 14 includes various non-limiting embodiments of chemical structures of polyfluorinated dicyclic compounds constructed in accordance with the present disclosure.

FIG. 15 includes various non-limiting embodiments of chemical structures of polyfluorinated dicyclic compounds constructed in accordance with the present disclosure.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/ device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The terms “analog,” “derivative,” or “variant” as used herein will be understood to refer to a variation of the normal or standard form or the wild-type form of molecules. For polypeptides, an analog may be a variant (polymorphism), a mutant, and/or a naturally or artificially chemically modified version of the wild-type polypeptide (including combinations of the above). Such analogs may have higher, full, intermediate, or lower activity than the normal form of the molecule, or no activity at all. Alternatively, and/or in addition thereto, for a chemical, an analog may be any structure that has the desired functionalities (including alterations or substitutions in the core moiety), even if comprised of different atoms or isomeric arrangements.

The term “hydrocarbon” as used herein will be understood to include any organic chemical compounds composed of carbon and hydrogen. The term “hydrocarbon” includes aliphatic (straight or branched chain) groups and/or aromatic hydrocarbon groups.

As used herein, “substantially pure” means an object species is the predominant species present (i.e., on a molar basis it is more abundant than any other individual species in the composition), and a substantially purified fraction is a composition wherein the object species comprises at least about 50 percent (on a molar basis) of all macromolecular species present. Generally, a substantially pure composition will comprise more than about 80 percent of all macromolecular species present in the composition, such as (but not limited to) more than about 85%, 90%, 95%, and 99%. In a particular (but non-limiting) embodiment, the object species is purified to essential homogeneity (contaminant species cannot be detected in the composition by conventional detection methods), wherein the composition consists essentially of a single macromolecular species.

Turning now to the inventive concept(s), certain non-limiting embodiments of the present disclosure are directed to a composition that comprises at least one polyfluorinated compound formed from two cyclic compounds (wherein each starting reactant comprises at least one aromatic group).

The term “polyfluorinated” as used herein will be understood to refer to a compound that includes at least two fluorines; however, the term “polyfluorinated” is not limiting of any other elements that may be present in the compound. That is, the polyfluorinated compound may actually be a polyhalogenated compound and thus include one or more other halogens (i.e., chlorine, bromine, and/or iodine). In addition, in a particular (but non-limiting) embodiment, the polyfluorinated compound may be a perfluorinated aromatic compound or a perhalogenated aromatic compound; in a perfluorinated aromatic compound, all of the hydrogens of the arene ring have been replaced with a fluorine, while in a perhalogenated aromatic compound, all of the hydrogens of the arene ring have been replaced with a combination of fluorine and one or more other halogens.

The term “polyfluorinated dicyclic compound” is used herein to indicate that the compound has at least two cyclic groups present (one from each starting reactant). However, it will be understood that the reference to “dicyclic” in the term “polyfluorinated dicyclic compound” simply indicates that the core structure of the compound comprises two cyclic groups, with one group obtained from each of the starting reactants (the core of each of which comprises an aromatic group), and does not limit the compound to only having two cyclic groups present; that is, each of the starting reactants may have one or more additional cyclic groups present as part of an R group attached to the aromatic group, and therefore the scope of the term “polyfluorinated dicyclic compound” also includes (for example, but not by way of limitation) polyfluorinated tricyclic compounds, polyfluorinated tetracyclic compounds, polyfluorinated pentacyclic compounds, polyfluorinated hexacyclic compounds, polyfluorinated heptacyclic compounds, polyfluorinated octacyclic compounds, polyfluorinated noncyclic compounds, polyfluorinated decacyclic compounds, and the like.

In certain particular (but non-limiting) embodiments, the polyfluorinated compound is a polyfluorinated cannabinoid or cannabinoid-like compound.

In certain non-limiting embodiments, the polyfluorinated compound comprises the structure of Formula I:

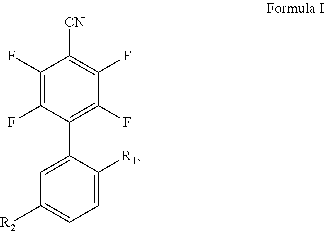

wherein R₁ is a hydroxy, ester, amide, ketone, cyanide, or piperidine group; wherein R₂ is H, a hydrocarbon, hydroxy, carbonyl, or butyl carbonate group; and with the proviso that when R₁ is methyl acetate, R₂ is not H or CH₃.

In a particular (but non-limiting) embodiment of the compound of Formula I, R₂ is a hydrocarbon selected from the group consisting of a methyl, ethyl, isopropyl, propyl, tert-butyl, pentyl, prenyl, and iso-prenyl group.

In another particular (but non-limiting) embodiment of the compound of Formula I, R₁ is methyl acetate. In this instance, R₂ may be selected from the group consisting of a hydroxy, methyl, ethyl, isopropyl, propyl, tert-butyl, pentyl, prenyl, iso-prenyl, carbonyl, and butyl carbonyl group.

In yet another particular (but non-limiting) embodiment of the compound of Formula I, R₁ is CN, and R₂ is CH₃.

In a further particular (but non-limiting) embodiment of the compound of Formula I, R₁ is N,N-di(propan-2-yl)acetamide, and R₂ is H or a hydrocarbon.

In certain non-limiting embodiments, the polyfluorinated dicyclic compound comprises the structure of Formula II:

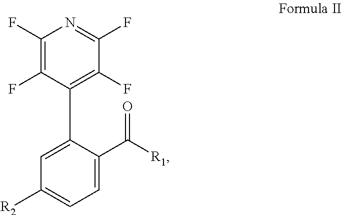

wherein R₁ is a hydroxy, ester, amine, amide, ketone, cyanide, or piperidine group; wherein R₂ is a hydrocarbon, hydroxy, carbonyl, or butyl carbonate group; and with the proviso that when R₁ is CH₃ or OCH₃, R₂ is not F or CH₃.

In a particular (but non-limiting) embodiment of Formula II, R₁ is OCH₃ or methylpiperidine, and R₂ is a hydrocarbon selected from the group consisting of a methyl, ethyl, isopropyl, propyl, tert-butyl, pentyl, prenyl, and iso-prenyl group.

In another particular (but non-limiting) embodiment of Formula II, R₁ is OCH₃, and R₂ is methyl acetate or a tert-butyl group.

In yet another particular (but non-limiting) embodiment of Formula II, R₁ is methylpiperidine, and R₂ is CH₃.

In certain non-limiting embodiments, the polyfluorinated compound comprises the structure of Formula III:

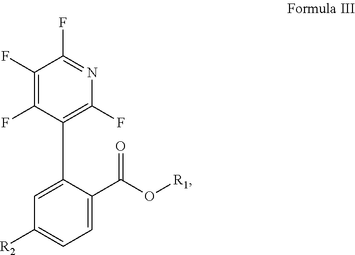

wherein R₁ is a hydroxy, ester, amide, ketone, cyanide, or piperidine group; wherein R₂ is H, a hydrocarbon, hydroxy, carbonyl, or butyl carbonate group; and with the proviso that when R₁ is CH₃, R₂ is not H.

In a particular (but non-limiting) embodiment of Formula III, R₁ is methyl acetate, and R₂ is CH₃.

In certain non-limiting embodiments, the polyfluorinated compound comprises the structure of the structure of Formula IV:

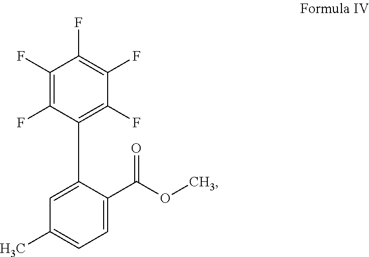

wherein R₁ is H, F, CF₃, a hydroxy, halogen, hydrocarbon, carbonyl, ester, amide, ketone, piperidine, butyl carbonate, or benzoate group; and with the proviso that R₁ is not CN, a hexanyl group, or a hexan-2-one group.

In a particular (but non-limiting) embodiment of Formula IV, R₁ is an ethyl, methyl acetate, or 2-hexanol group.

In another particular (but non-limiting) embodiment of Formula IV, R₁ is a hexan-2-yl 3,5-bis(trifluoromethyl)benzoate group.

In certain non-limiting embodiments, the polyfluorinated compound comprises the structure of the structure of Formula V:

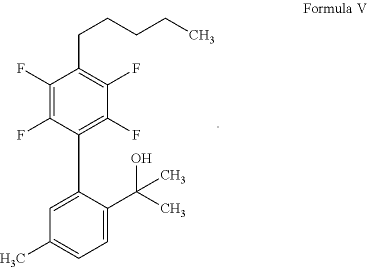 [SMILES: CCCCCc1c(F)c(F)c(-c2cc(C)ccc2C(C)(C)O)c(F)c1F]

In certain non-limiting embodiments, the polyfluorinated compound comprises the structure of Formula VI:

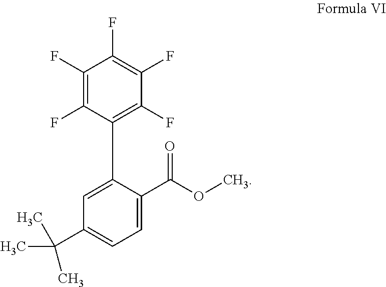 [SMILES: COC(=O)c1ccc(C(C)(C)C)cc1-c1c(F)c(F)c(F)c(F)c1F]

In certain non-limiting embodiments, the polyfluorinated compound comprises the structure of one of the stereoisomers of Formulas VII, VIII, and IX:

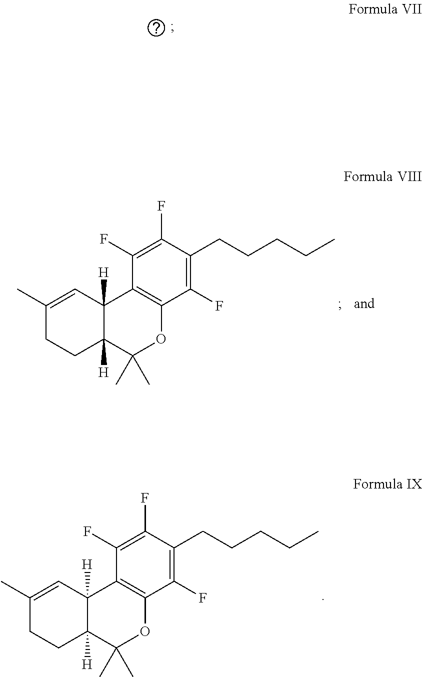

In certain non-limiting embodiments, the polyfluorinated compound comprises the structure of Formula X:

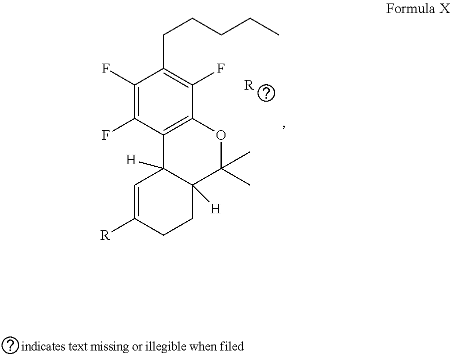

wherein R is H, a hydrocarbon, hydroxy, carbonyl, or butyl carbonate group.

In certain non-limiting embodiments, the polyfluorinated compound comprises the structure of Formula XI:

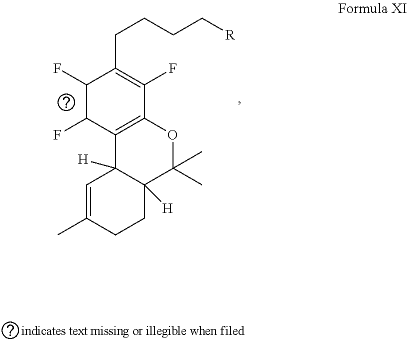

wherein R is a hydrocarbon. Non-limiting examples of hydrocarbons that may be utilized in accordance with the present disclosure include a C1-C3 hydrocarbon, an aromatic group, a phenyl group, a pentyl group, an alkane, and combinations thereof.

In certain non-limiting embodiments, the polyfluorinated compound comprises the structure of Formula XII:

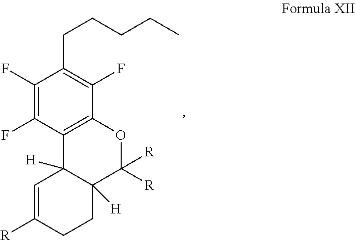

wherein each R is independently selected and is a hydrocarbon. Non-limiting examples of hydrocarbons that may be utilized in accordance with the present disclosure include a C1-C3 hydrocarbon, an aromatic group, a phenyl group, a pentyl group, an alkane, and combinations thereof.

In a particular (but non-limiting) embodiment, each R is an alkane.

In certain non-limiting embodiments, the polyfluorinated compound comprises the structure of Formula XIII:

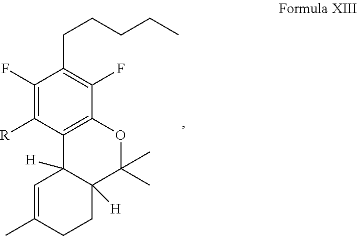

wherein R is F, OH, OR′, SH, SR′, NH₂, or NHR′.

In a particular (but non-limiting) embodiment of Formula XIII, R is F, and the compound is a specific stereoisomer thereof.

In certain non-limiting embodiments, the polyfluorinated compound comprises the structure of Formula XIV:

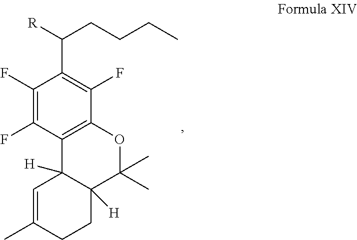

wherein R is a hydrocarbon.

In a particular (but non-limiting) embodiment of Formula XIV, R is an alkane.

Certain non-limiting embodiments of the present disclosure are directed to compositions that are mixtures of two or more of any of the polyfluorinated compounds disclosed or otherwise contemplated herein. For example, but not by way of limitation, the composition may contain a mixture of about two, about three, about four, about five, about six, about seven, about eight, about nine, about ten, about 11, about 12, about 13, about 14, about 15, about 16, about 17, about 18, about 19, about 20, or more of the polyfluorinated compounds disclosed or otherwise contemplated herein, as well as a range of two of any of the values above (i.e., a mixture comprising a range of from about two to about 20 polyfluorinated compounds, etc.).

Certain non-limiting embodiments of the present disclosure are directed to a method of synthesizing any of the polyfluorinated compounds as described or otherwise contemplated herein.

Certain non-limiting embodiments of the present disclosure are directed to a method of synthesizing a polyfluorinated cannabinoid or cannabinoid-like compound. In certain particular (but non-limiting) embodiments, the method utilizes a photocatalytic Birch-type reduction. The individual method steps may be performed as described in any of the Examples, which are expressly incorporated herein by reference. In addition, while the Examples may describe synthesis reactions for one or more particular compounds, it will be understood that these same synthesis reactions will be applicable to the synthesis of other related compounds. For example (but not by way of limitation), while a particular synthesis reaction disclosed in one of the Examples produces one compound of Formula I with particular R₁ and R₂ groups, the same or similar reactions may be utilized for the production of another compound of Formula I with different R₁ and R₂ groups. Therefore, methods of producing all of the compounds of Formulas I-VI fall within the scope of the present disclosure.

Certain non-limiting embodiments of the present disclosure are directed to a method that comprises combining a polyfluorinated aromatic compound with an aromatic compound having at least one activated group to form a mixture, and reacting the mixture under conditions that result in coupling with concomitant dearomatization to form a polyfluorinated compound having at least a dicyclic core structure (such as, but not limited to, a cannabinoid derivative).

In certain particular (but non-limiting) embodiments, the polyfluorinated aromatic compound has a structure of the following formula:

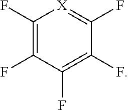

In a particular (but non-limiting) embodiment, X is N, and therefore the polyfluorinated aromatic compound is a polyfluorinated pyridine. In another particular (but non-limiting) embodiment, X is C—R, wherein R is an electron withdrawing group. For example (but not by way of limitation), the R group may be a nitrile group, a trifluoromethyl group, a pentanal group, a methyl group, a methyl ester group, a pentyl group, a carbonyl group, a sulfone group, an alkyne group, and the like.

Alternatively, in other particular (but non-limiting) embodiments, the polyfluorinated aromatic compound has a structure of the following formula:

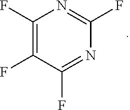

Alternatively, in yet other particular (but non-limiting) embodiments, the polyfluorinated aromatic compound has a structure of the following formula:

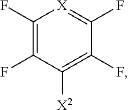

wherein X is N, CF, or C—R, wherein R is an electron withdrawing group as described herein above; and X² is Cl or Br.

In certain particular (but non-limiting) embodiments, the polyfluorinated aromatic compound is a polyfluorinated pyridine, a polyfluorinated pyrimidine, or a polyfluorinated benzonitrile.

In a particular (but non-limiting) embodiment, the polyfluorinated aromatic compound is 1-(perfluorophenyl)pentan-1-one.

In a particular (but non-limiting) embodiment, the polyfluorinated aromatic compound comprises 1-(trifluoromethyl)-2,3,5,6-tetrafluorobenzene.

In certain particular (but non-limiting) embodiments, the aromatic compound with at least one activated group has a structure of the following formula:

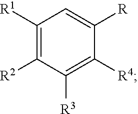

wherein R is an electron withdrawing group; and each of R¹, R², R³, and R⁴ is independently selected from H, a hydrocarbon group having between 1-20 carbons, an aromatic group, or O with a suitable protecting group (such as, but not limited to, a BocO, methyl carbonate, or acetate (such as, but not limited to, a polyvinyl acetate (Pivlate)) group).

For example (but not by way of limitation), R may be a methyl ester group, and each of R¹, R³, and R⁴ may be H. R² may then be selected from the group consisting of H, O, OH, CH₃, a tert-butyl group, a methyl ester group, or a BocO group.

In certain particular (but non-limiting) embodiments, the method may include one or more additional steps. One non-limiting example of an additional step includes adding water to the mixture.

Each of the methods described or otherwise contemplated herein may produce the polyfluorinated compounds with any level of yield. For example (but not by way of limitation), the polyfluorinated compounds can be synthesized with a yield of at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, and at least about 99%. In addition, the scope of the presently disclosure also includes the production of the polyfluorinated compounds at any percent yield that falls within any range formed from the combination of two values listed above (for example, a range of from about 1% to about 99%, a range of from about 10% to about 98%, a range of from about 30% to about 97%, a range of from about 50% to about 96%, a range of from about 60% to about 95%, etc.).

Certain non-limiting embodiments of the present disclosure are directed to a polyfluorinated compound produced by any of the methods described or otherwise contemplated herein.

### EXAMPLES

Examples are provided hereinbelow. However, the present disclosure is to be understood to not be limited in its application to the specific experimentation, results, and laboratory procedures disclosed herein. Rather, the Examples are simply provided as one of various embodiments and are meant to be exemplary, not exhaustive.

### Example 1

As stated herein above in the Background section, fluorination has assumed a lead role in discovery chemistry because of its ability to improve a host of properties in a multitude of applications. The importance of fluorination in pharmaceuticals can be observed in that typically 30% of all drugs approved per annum by the U.S. FDA contain a C—F bond; in 2018, of the small molecule entities that were approved by the U.S. FDA, 17 contained a C—F bond, 12 were fluoroarenes, and 3 were polyfluorinated arenes, indicating that arene polyfluorination specifically is becoming an increasingly important synthetic objective.

Despite the importance of fluorinated molecules in the pharmacopeia, linear syntheses of fluorine-containing molecules have proven difficult. Previously used sequential fluorine installation methods such as the Balz-Schiemann and halex reactions are difficult and not typically functional group tolerant. In addition, while recent developments have increased the number of functional groups that can be transformed into fluorine, these techniques are only useful for monofluorination and contribute little in the exploration of multifluorinated molecules, as a library of multi-site prefunctionalized non-fluorinated starting materials would be required.

Recently, the synthesis of organofluorines has been approached from the other way around (Capdevila et al., 2019; Luo et al., 2018). Rather than approaching fluorination through harsh, multi-step, or low yielding linear syntheses, the desired organofluorine can be realized by starting with all of the fluorines already in place, with poly- or perfluorinated core molecules, and replacing the unwanted fluorines through hydrodefluorination (HDF) (Senaweera et al., 2014; Chen et al., 2017; Vogt et al., 2019; Matsunami et al., 2018; Lu et al., 2016) or turning them into a desirable substituent through direct alkylation, alkenylation, arylation, and prenylation (Luo et al., 2018; Singh et al., 2015; Singh et al., 2016; Senaweera et al., 2016; Nicholls et al., 2018; Cao et al., 2019; Dai et al., 2018; Meyer et al., 2016). Visible light photocatalysis with an iridium based photocatalyst has played a critical role in this area, and the interplay between photocatalytic C—F functionalizations and other methods has the potential to allow for sophisticated, but as of yet, largely unrealized SAR with respect to fluorine. Furthermore, the mild nature of these reactions may lend itself to the formation of unnatural products—fluorinated natural products, which could create new opportunities to explore the effect of fluorination on the function of natural products, which is a mostly unexplored domain.

Previously, inventors Senaweera and Weaver have shown that it was possible to couple a perfluoroarene directly with an arene partner via C—F and C—H functionalization (Senaweera et al., 2016). During this investigation, careful assessment of some of the minor products in the crude reaction NMR spectra revealed vinylic ¹H signals, and GC-MS analyses showed the [M+2] peak with respect to the expected product, a highly unexpected result. It was expected that a cyclohexadienyl radical was a likely intermediate in the mechanism for the fluoroaryl arylation reaction, and that while the majority of the materials were proceeding through a mechanism in which they rearomatized—formally losing an H-atom in the process. Under certain conditions, the reduction of that intermediate had in fact gained an H-atom, resulting in what amounts to a formal hydrogenation of the intended products.

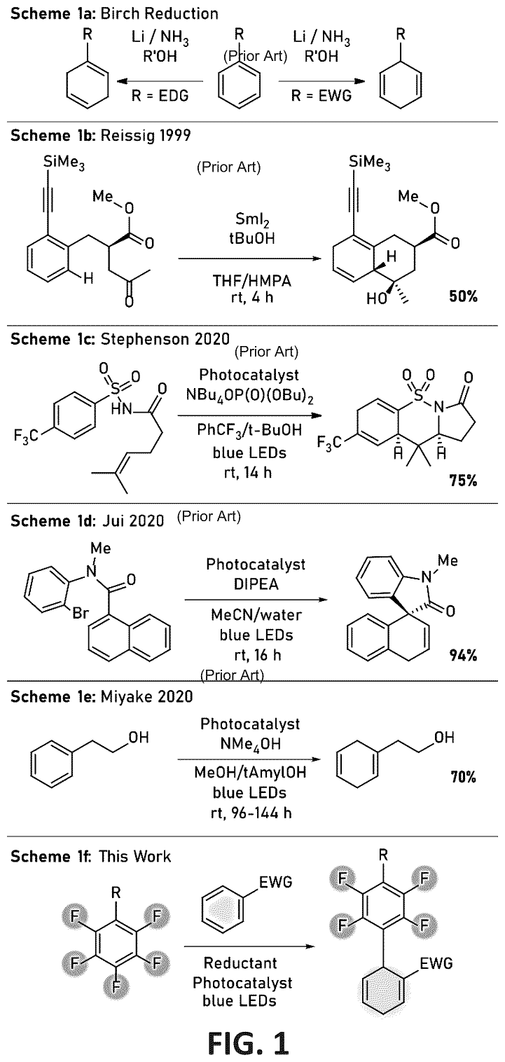

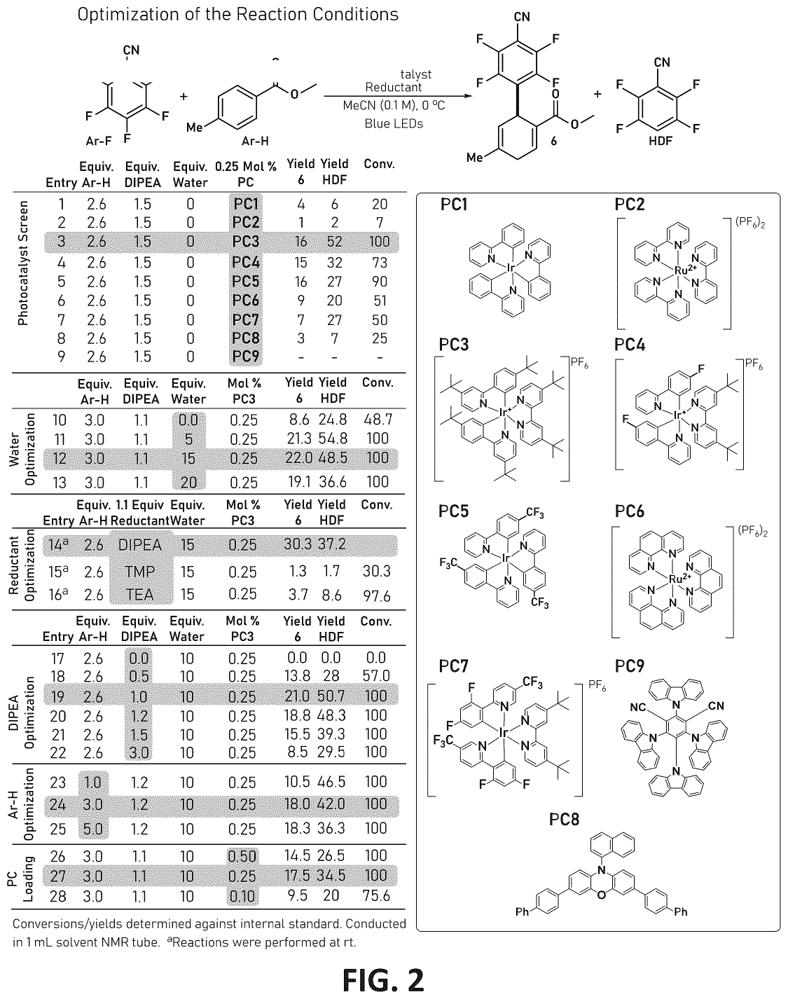

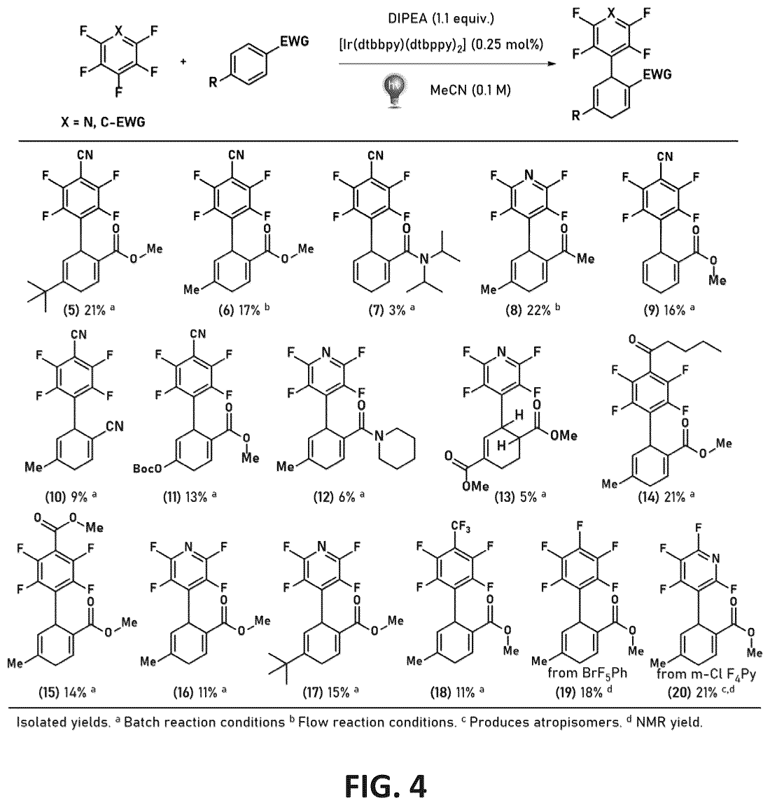

After careful isolation, simulated NMR spectra were generated of all possible perturbations of diene geometry for comparison to the experimental spectra, using the method of Bally and Rablen (2011). Of these, several contenders were similar to the experimental spectrum of 8, although the 1,4-diene depicted in Scheme 1b of FIG. 1 appeared to most accurately match the experimental spectra. Seeking more concrete data, a crystal was grown and submitted for single crystal X-ray diffraction analysis, which confirmed the structure (FIG. 4, structure 8). This product is structurally related to the product of a Birch (1950) type dissolving metal reduction (FIG. 1, Scheme 1a). However, in addition to the reduction, it simultaneously formed a new C—C bond. Another key difference is the regioselectivity of the diene, which differs from those formed under Birch conditions for substrates possessing an electron withdrawing group (FIG. 1, Scheme 1a) (Birch, 1950; Chapman et al., 1963). The conditions used for the Birch reaction (alkali metals in condensed liquid ammonia) are harsh and severely limit the scope of the reaction, while the reactions presented here proceed in much milder conditions, and could be expected to be significantly more functional group tolerant. Work by Reissig demonstrated the ability of ketyl radical to undergo dearomative cyclization using stoichiometric Sm(II) (McAtee et al., 2019). It is important to note that during the exploration of this methodology, a few photocatalytic dearomative methods have surfaced in the literature (McAtee et al., 2019; Dong et al., 2020; Gao et al., 2009), but the reaction discussed herein (FIG. 1, Scheme 1f) is unique in that it forms a C—C bond but does not require the pieces to be tethered together, nor is it a spirocyclization, and it abstracts hydrogen (formally) from water (Dong et al., 2020). The intermolecular nature of the reaction may facilitate SAR studies in ways that intramolecular reactions cannot. While not being bound by any particular theory, it was considered that, because of the structural complexity and functional richness of these dienes, it would be a valuable asset to the chemical community and set out to discover optimized conditions (FIG. 2) which would favor the formation of the reduced 1,4-diene.

Optimization:

Searching for optimal reaction conditions revealed that the mass balance for the products of the reaction, apart from several major products consisted of a complex mixture of oligomers. Upon screening photocatalysts (FIG. 2, entries 1-9), it was discovered that a somewhat oxidizing (Lowry et al., 2004; Teegardin et al., 2016), sterically bulky photocatalyst that has been thus far relatively underrepresented in the literature, bis-4-(tert-butyl)-2-(4-(tert-butyl)phenyl)pyridine-4,4′-di-tert-butyl-2,2′-bipyridyl Iridium(III) hexafluorophosphate (Ir(dtbbpy)(dtbppy)₂PF₆) (PC3), with a triplet state emissive energy of 49.4 kcal/mol (Daub et al., 2019), E⁰(Ir^{II}/Ir^{III}*)=−1.04 V, and E⁰(Ir^{II}/Ir^{III})=+1.13 V42, was optimal. While PC5 had a similar outcome in terms of the intended product, the reaction produced many minor side products.

Recently, Chaterjee and Koenig (2019) disclosed a dearomatization that is proposed to take place by triplet energy transfer followed by SET. However, because molecular radius of the photocatalyst has been shown to be critical for energy transfer to occur (Singh et al., 2016), the presence of large tert-butyl groups on this photocatalyst likely makes the probability of the necessary orbital overlap of the photocatalyst and the substrate unlikely (Singh et al., 2018). Moreover, given the relatively low triplet state energy of (Ir(dtbbpy)(dtbppy)₂PF₆), (49.4 kcal/mol) (Singh et al., 2016; Day et al., 2018), it is possible that energy transfer may not be operative.

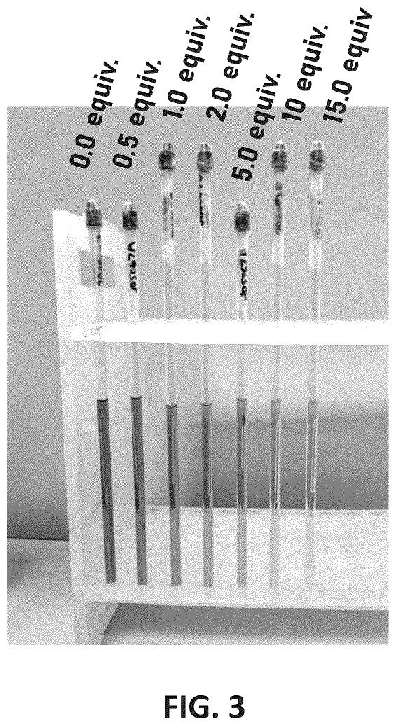

It was discovered that the presence of water significantly enhanced the reaction outcome (FIG. 2, entries 10-13). Further, the addition of water caused the reaction to remain a pale yellow color, while in the absence of water, it darkens significantly. It could be that the presence of water further stabilizes a charged intermediate or transition state, or it could be serving to stabilize and solvate the fragmenting fluoride, a potentially very exothermic process (see discussion of the mechanism below). It could also be serving a more abstruse role, such as preventing the formation of light absorbing compounds that could lead to photostarvation if formed (FIG. 3; Rathnayake et al., 2020). The screening for the identity of terminal reductants revealed tributylamine and diisopropylethylamine (FIG. 2, entries 14-16) to be nearly identical, while all other reductants attempted gave poor or no product. DIPEA was utilized due to its comparatively increased vapor pressure and water solubility, which was anticipated would facilitate product isolation. A precipitous decline in yield of the intended diene was observed on either side of the optimized stoichiometry (1.0-1.2 equiv.) with respect to the amount of terminal reductant (FIG. 2, entries 17-22). Hydrocarbon arene coupling partner (Ar—H) equivalents were found to be optimal at 3.0 (FIG. 2, entries 23-25). Photocatalyst loading (FIG. 2, entries 26-28) was found to be optimal for an NMR tube at 0.25 mol %, but product distribution was not affected at lower loadings, with the reaction simply requiring more time to reach completion. Upon scaling the reaction up, the increased path length proved to slow the reaction rate, though the catalyst loading could be decreased to 0.01 mol % without decreasing the yield. It was further found that the reaction depends heavily on reaction solvent, with poor or no reactivity in any solvent tried except acetonitrile. The reaction temperature (−2° C. to 50° C.) was found to have little impact on the yield, allowing subsequent reactions to be run at convenient temperatures. The optimization experiments provided insight into the influence of the various parameters; however, yields remained meager. Additionally, under no conditions found could the minor side products, which were significant and ill-defined, be completely diminished. However, given the commercial availability of all the reagents, the operational simplicity of the reaction, and the rapid enhancement of structural complexity of the reaction, the scope was investigated.

The reaction operates in a number of different fluoroarenes and coupling partners (FIG. 4). Reactions in which varying the Ar—H with benzoate esters (structures 5, 6, 9, 11, 13, 14, 15, 16, 17, 18, 19, and 20), ketones (8), benzonitriles (10), and amides (7, 12) progressed nicely, though in lower yields. In addition, separation of the material from the reaction mixture proved difficult, especially for those reactions in which the Ar—H was not appreciably volatile, such as the benzamides, and thus could not be separated via distillation. Variation of the fluoroarene revealed that a wide variety of fluoroarenes operated similarly, although variation of the Ar—H from 4-methyl (6) to 4-tertbutyl (5) benzoates resulted in somewhat better yields or easier separations. Interestingly, reaction with a bromopentafluorobenzene resulted in the typical 1,4 diene product connected at the position formerly occupied by the bromine (19). This is interesting because it serves as a mechanistic probe, suggesting the involvement of an aryl radical (see discussion below). The reaction with 3-chloro-2,4,5,6-tetrafluoropyridine also undergoes chemoselective fragmentation of the chloride, superseding the regioselectivity for the 4-position to yield the dihydro product (20), and reveals an interesting property of the products. The bicyclic motif is rotationally locked into a conformation producing atropisomers which do not interconvert at room temperature (Kumarasamy et al., 2015). While likely true for all substrates, it is not evident with symmetrical fluoroarenes. In this case, the C—C bond formation gives rise to both axial and point chirality with a slight preference for one diastereomer (2.4:1). These diastereomers can be partially separated by recrystallization with the minor diastereomer becoming enriched (1:1.9). Under these conditions, the reaction operates with all of the fluoroarenes shown here, reactions attempted with simple perfluorobenzene did not react at all, indicating that the fluoroaryl reduction potential (−2.11 V in DMF vs SCE) (Lu et al., 2016; Loutfy et al., 1976) is outside that reachable by the photocatalyst. Additionally, when 4-iodobenzonitrile, devoid of fluorine, was subjected to the reaction conditions with 4-methyl acetophenone, only trace amounts of the coupled products (both diene and biaryl) were detected by GCMS; instead, hydrodeiodination was the major product. This suggests that the fluorine substituents, when compared to the defluoroarene, facilitate the C—C bond formation. While yields are generally modest, this reaction provides a rapid increase in complexity from commercially available starting materials and scales well. Compound 6 was run on a 10 mmol scale and produced 553 mg of fluorinated diene, and 947 mg diene 14 was produced from a 12.0 mmol scale batch reaction according to general procedure B, which was sufficient for downstream synthetic manipulations. Reactions could be scaled using either high intensity LEDs and tubes with the addition of a stir bar, or in a flow reactor setup.

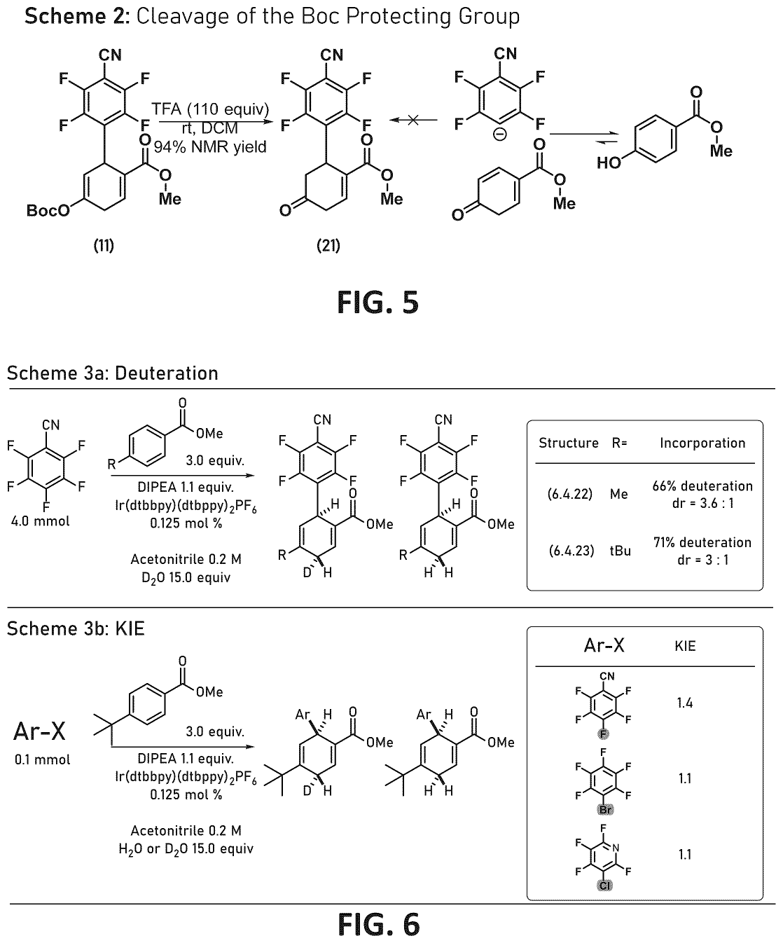

While phenols are largely incompatible with these conditions, Boc-protected methyl paraben was investigated and found to be a competent coupling partner. Upon cleavage with TFA, this reaction provides a keto-cyclohex-enyl methyl carboxylate. The two-step process formally provides access to the product of Michael addition with the keto-tautomer of methyl paraben (FIG. 5 (Scheme 2), right). With the five sites of reactive functional groups, it is both functional group dense, and a structure that would be extraordinarily difficult to reach given other methods. Such functionally dense structures should allow facile access to more complex classes of molecules that can facilitate further synthetic manipulations.

While not wishing to be bound by any particular theory or mechanism, the following discussion of potential, non-limiting mechanisms involved are provided herein below.

While the selectivity of the reaction involves C—F fragmentation selectivity, C—C bond formation regioselectivity, and C—H bond formation regioselectivity, and in certain cases-modest atropselectivity, it is remarkably predictable, forming only a single dearomatized isomer. The halogen selectivity follows trends previously observed in photocatalytic C—F functionalization. The 4-position of any monosubstituted perfluoroarene preferentially fragments (Shchegoleva et al., 1999), and when present, heavier halogens preferentially undergo fragmentation (FIG. 4, structures 19 and 20) (Senaweera et al., 2014 and 2016; Singh et al., 2015; Meyer et al., 2016). The regioselectivity of the C—C bond formation between the fluoroaryl fragment and the hydrocarbon aryl coupling partner appears to be dictated by the LUMO of the Ar—H partner. The reaction is selective for the carbon ortho to an electron withdrawing group and works best for substrates in which the predicted LUMO is larger at the ortho position and significantly smaller elsewhere. Arenes lacking electron withdrawing groups fail to give any cyclohexadiene product, suggesting that the electron withdrawing functional group may play other critical roles. At this time, the driving forces that lead to the observed diene regioisomer are unclear.

As mentioned above, water played a critical role in the reaction. Darkening of the reaction mixture could be avoided by the addition of water (FIG. 3). This accompanied much welcomed improvements in product distribution (FIG. 2) and a reduction of the number of minor side products formed. This might be explained by the formation of cyanine dyes arising from oligomerization of the DIPEA, as those dyes are known to form under similar conditions and known to participate in photocatalytic reactions. The inclusion of water may prevent their formation, or hydrolyze them if they do form (Rathnayake et al., 2019; Böhm et al., 2016).

It was originally presumed that the amine indirectly (as the radical cation) served as the terminal reductant and source of H-atom in the formation of the diene. However, it was found that when normal water was replaced with heavy water (15 equiv D₂O, FIG. 6, Scheme 3a), the reaction resulted in 69% deuterium incorporation into the diastereotopic methylene signal. Resubjecting the isolated product (22 or 23) to reaction conditions with the inclusion of D₂O rather than H₂O showed that post-reaction exchange was not operative (slow HDF was observed). Furthermore, the deuterium incorporation occurred with a ca. 3:1 (75%) selectivity on the side opposite the perfluoroaryl ring. These findings obviate the possibility of the reaction proceeding solely through a hydrogen atom transfer (HAT) process, because the bond dissociation energy of water at 117.9 kcal/mol (Beatty et al., 2015) is far from the weakest bond (BDE for cyclohexadiene ca. 77 kcal/mol (Gao et al., 2009), ca. 90 kcal/mol for neutral DIPEA, and ca. 42 kcal/mol as the DIPEA amine radical cation) (Beatty et al., 2015; Wayner et al., 1986), placing water well outside the realm of reasonable H-atom sources. It is more likely that the reaction proceeds through either an anionic reaction mechanism similar to the traditional Birch reaction, or through a proton coupled electron transfer (PCET) event in which the cyclohexadienyl radical is reduced to the carbanion and protonated. Because deuteration is incomplete, however, it is possible that the reaction is proceeding through both an anionic pathway and an HAT event (Costentin et al., 2019). Attempts to identify more acidic or nonaqueous additives instead of water (isopropanol, ethanol, trifluoroethanol) that would favor the formation of the intended product proved deleterious to the reaction.

Upon further investigation, a kinetic isotope effect (KIE) was observed in parallel experiments where either H₂O or D₂O were included in the reaction mixture, with a k_{H}/k_{D} of 1.4 (FIG. 6, Scheme 3b, Eqn. 1), which is consistent with a secondary KIE, or a solvent KIE. This indicates that the rate-determining step (RDS) is not protonation of an anionic intermediate by water, nor is it a PCET event, steps for which a larger KIE could be expected (Huynh et al., 2007). One potential explanation could be that there is a pre-rate determining equilibrium, and that the protonation of the carbanionic intermediate is near the rate determining step. Alternatively, it could also be explained by fragmentation of a fluoride as the RDS. The observed KIE would result from the solvation of the fragmenting fluoride (Montanari et al., 1993), a generally exothermic step by ca. 104.4 kcal/mol, could be expected to affect the kinetics of this step (Zhan et al., 2004; Kelly et al., 2006). By comparison the solvation of chloride and bromide are generally less exothermic, (74.5 and 68.3 kcal/mol respectively), and in general undergo faster mesolytic fragmentation (Kelly et al., 2006). Reactions with either bromopentafluorobenzene or 3-chloro-tetrafluoropyridine (FIG. 6, Scheme 3b, Eqns. 2 and 3, respectively) lead to a 1,4-diene product by fragmenting a non-fluorine halogen. Initial rate studies with these substrates indicate a KIE of 1.1, much less than analogous substrates in which a C—F bond is functionalized. This suggests that for the substrates from which fluoride is fragmented, the RDS is the mesolytic fragmentation of the radical anion.

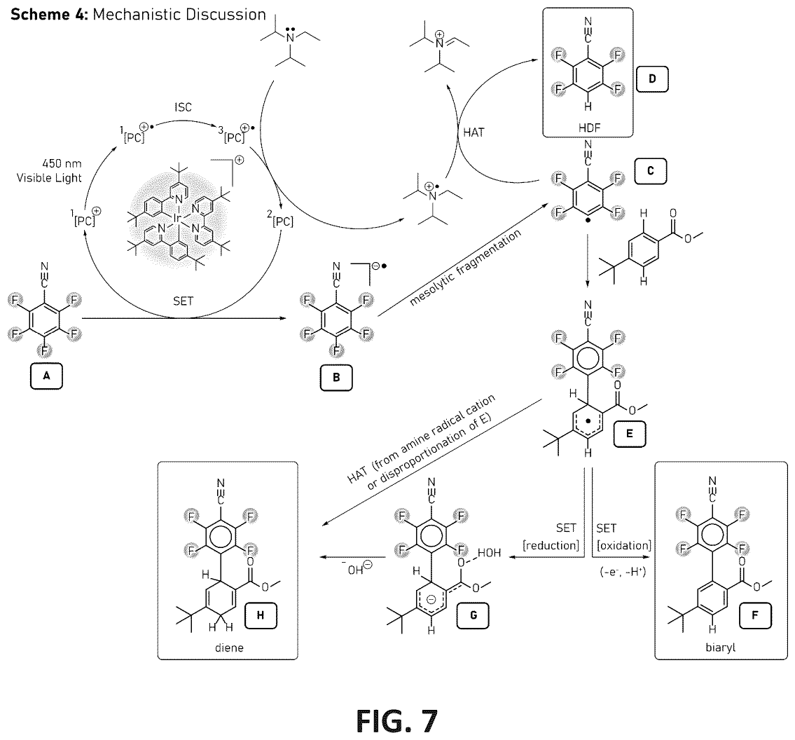

Taking the above experiments and observations, the working mechanistic understanding is shown in Scheme 4 (FIG. 7). After absorption of a photon an initial singlet photocatalyst rapidly undergoes inter-system crossing to yield the triplet excited state photocatalyst (Flamigni et al., 2007; Campagna et al., 2007). Based on redox potentials, the reaction is expected to proceed through a reductive quenching cycle, in which the triplet photocatalyst (E⁰(Ir^{II}/Ir^{III}*)=−1.04 V42) is reduced by an amine (Eₒₓ ca. 0.5 V vs. SCE (McTiernan et al., 2016). The reduced photocatalyst then undergoes SET to the fluoroarene (A) to form the radical anion (B), and return the photocatalyst to the ground state. In the RDS, this radical anion, aided by water, fragments mesolytically to give the fluoroaryl radical (C) and a fluoride anion (Senaweera et al., 2014 and 2016; Singh et al., 2015). Based on BDEs, the fluoroaryl radical is expected to easily abstract an H-atom from DIPEA, or its corresponding radical cation, to give the major byproduct, the hydrodefluorinated product (D).

One might expect that the fluoroaryl radical anion (B, Scheme 4 of FIG. 7) with its abundance of electron density could potentially act nucleophilically (Bertran et al., 1996; Smothers et al., 1979), attacking the LUMO of the Ar—H to produce an intermediate distonic radical anion. While there are reports of nucleophilic attack by related radical anions (Smothers et al., 1979), it seems unlikely in this reaction. This is due to the observation that both chlorinated and brominated fluoroarenes lead to the same 1,4-diene product, the observation of a rate enhancement upon increase of H₂O, and the observation that the KIE is significantly retarded in the presence of a fragmenting bromide or chloride, all of which are consistent with fragmentation of the halogen being rate determining. In other words, the rate of halogen fragmentation increases as the size of the halogen increases and the corresponding radical anions are less reliant on solvation (compared to fluoride) to facilitate fragmentation. Consequently, the k_{H}/k_{D} diminishes as the rate of fragmentation increases, barring a mechanistic change between these substrates. This rate could be expected to be much more rapid upon forming an intermediate distonic radical anion. A solvent KIE, if observed at all under such conditions, would be the same for all substrates regardless of the identity of the fragmenting halogen.

It is therefore more likely that, as with previous photocatalytic reactions reported, that the reaction occurs through the mesolytic fragmentation of the radical anion (B, Scheme 4, FIG. 7) to form the fluoroaryl radical (C), which then attacks the LUMO of the Ar—H, leading directly to (E). The partial incorporation of protium may be best explained by a mechanistic bifurcation from intermediate E. Therefore, it is proposed that the delocalized, doubly allylic radical (E) is itself either reduced by the photocatalyst to give an anionic intermediate (G) (Abbas et al., 2018) which provides the observed 1,4-diene (H) upon protonation or oxidized to give the other observed major byproduct, the rearomatized biaryl product (F). It is also possible that the formation of the biaryl and the intended dienyl product originate from disproportionation reaction of the radical E, in which the cyclohexadienyl radical abstracts an H-atom from another cyclohexadienyl radical E to form both the observed reduced product (BDE ca. 77 kcal/mol for unsubstituted substrate) (Gao et al., 2009) and oxidized biaryl. Although this pathway fails to account for the incorporation of the deuterium from water, again the deuteration is incomplete. The amount of biaryl side product is always less than the amount of 1,4-diene, which supports (but is not limited to) a mechanistic bifurcation.

Application:

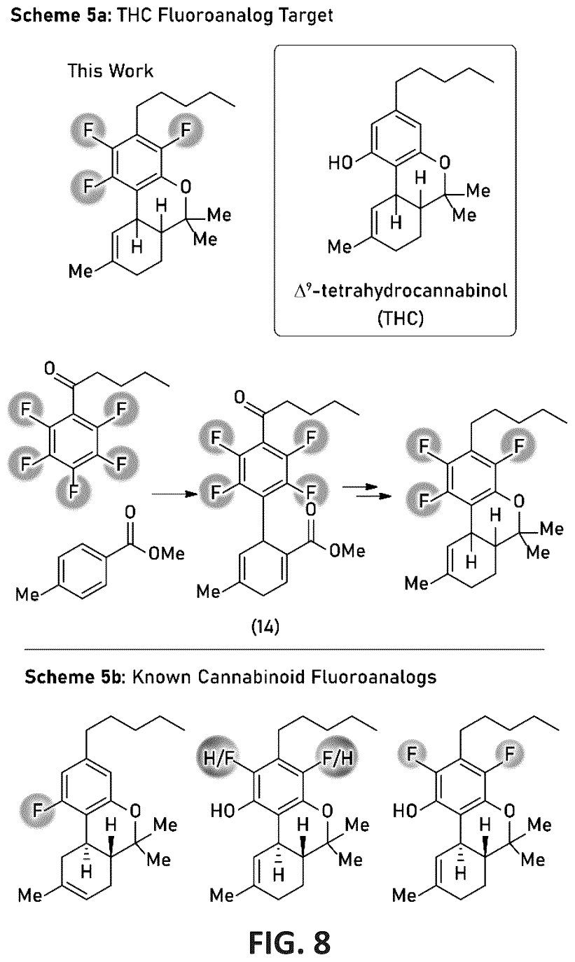

Returning to our original goal of enabling the synthesis of unnatural products, it was observed that this motif maps very nicely onto the structural skeleton of classical cannabinoids (FIG. 8, Scheme 5a), the most prominent, and also most notorious of which is trans-Δ⁹ -tetrahydrocannabinol (THC) (Banister et al., 2019), the molecule not only responsible for most of the psychoactive effects of cannabis, but also many of its medicinal properties.

Despite the extensive and ongoing research on classical cannabinoids, F3-THC has, to the best of our knowledge, not been reported before. Related compounds with mono- or di-fluorination (Martin et al., 2002; Westphal et al., 2017) are known in the literature (Breuer et al., 2016; Banister et al., 2015) and have been shown to have bioefficacy (FIG. 8, Scheme 5b). This method allows access to a completely unknown series of trifluoro analogs with substitution at either the Ar—F or the Ar—H for diversification through a straightforward series of reactions and importantly will allow us to investigate the role of the hydroxyl group on THC.

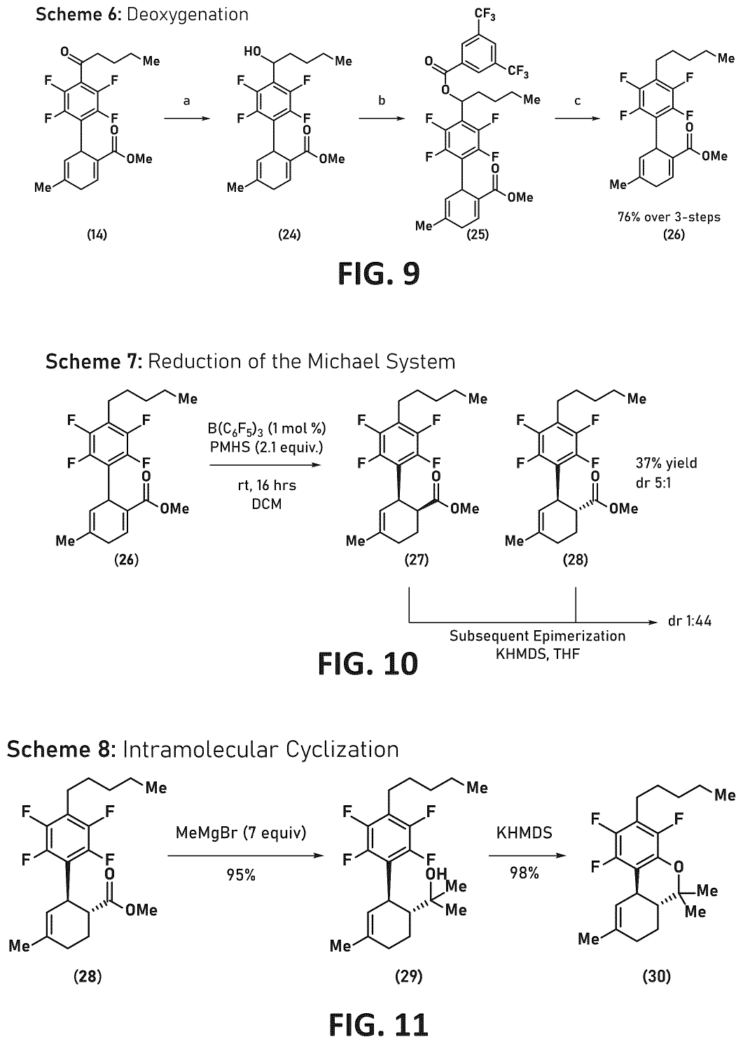

For the synthesis of F₃-THC, diene (14) (FIG. 8, Scheme 5a) was chosen as one of the most suitable starting materials, because reactions toward a more direct route with the simple pentyl alkyl pentafluoroarene produced none of the intended intermediate materials, presumably because the reduction potential is too high with an electron donating pentyl group. Following the photo-Birch reaction, methods for the deoxygenation of the aromatic ketone were investigated. Following a clean reduction of the ketone with NaCNBH₃, compound 24 (FIG. 9, Scheme 6) was obtained in high yield. The Barton-McCombie approach (1975) was found to be attractive, and the elegant photochemical approach developed by Reiser et al. (Rackl et al., 2014) was pursued, which provided the intended deoxygenated product in three straightforward steps (Rackl et al., 2014; Lau et al., 1986) with a total yield of 76%.

Following the successful formation of the desired intermediate diene (26), reduction of the Michael system was addressed. This proved to be remarkably difficult. This was surprising given the expected electronic and steric differences between the alkenes, and yet, using a number of more common methods, unselective reaction, unintended side products, or poor conversion were observed. The most reliable method was found to be a derivative of the method by Chandrasekhar et al. (2006), hydrogenation via polymethylhydrosiloxane (PHMS) with a strong Lewis acid, tris(pentafluorophenyl)borane (FIG. 10, Scheme 7), which ultimately gave the hydrogenated product in modest yields and in a diastereomeric ratio of around 5:1 (varying between 4:1 and 6:1) in favor of the cis product (27) over the trans product (28). The desired epimer 28, could be achieved by the use of stoichiometric KHMDS in THF to yield a 1:44 diastereomeric ratio in favor of trans 28. The trans diastereomer was desirable in part as natural (trans) THC has a higher affinity for cannabinoid receptors than its cis counterpart. It could be reasonably expected that the fluorinated analogs would perform similarly.

Having determined a route to the trans intermediate cyclohexene (28), the formation of the bridging ring was then investigated (FIG. 11, Scheme 8). It was initially desired to be able to form the third ring through tandem nucleophilic addition of the methyl groups followed by subsequent S_{N}Ar displacement of the fluoride. Unfortunately, this did not prove fruitful under the conditions tested with either methyl Grignard or lithiate. Of note, while the cis and trans cyclohexenyl methyl esters (27 & 28) were challenging to separate, upon conversion to the corresponding cis- and trans-alcohols (29) showed different R_{f} values and could be more easily separated chromatographically. However, treating alcohol (29) with KHMDS afforded cyclization, similar to the work by Westphal et al. (2017), and provided the intended cannabinoid product (30) with a 93% yield over 2 steps (FIG. 11, Scheme 8). Ultimately, the reaction starts with two commodity chemicals and takes place in 8 succinct steps with 6% overall yield. Furthermore, several points of diversification exist and could be used for further exploration of the motif, and several fluorinated cannabinoids are bioassayed.

Conclusion:

A new reaction has been demonstrated that provides a new and expedient route to 1,4-dienes, that are a tremendous advancement for medicinal discovery chemistry because of the rapid enhancement in chemical complexity. Further, it has been shown that the reaction likely proceeds through an anionic intermediate, which opens the door for potential further diversification through a Birch-like alkylation. These dienes map readily onto the carbon framework of classical cannabinoids, which have been shown herein can be synthesized in short order, which would otherwise have previously been prohibitively difficult to reach. In addition, the synthetic steps offer a wealth of synthetic possibilities for diversification. Further, the synthesis laid out herein can provide access to fluorinated analogs of existing CB1/CB2 agonists such as classical THC, related cannabinoids, or perrottetinenes. In addition, one of the major byproducts, the rearomatized biaryl species could lead to additional analogs of the natural cannabinoid cannabinol (CBN). Coupled with existing defluorination techniques and the possibilities present for downstream diversification, the possibilities for more complete SAR with respect to fluorination are possible. The reactivity shown herein is previously unknown and is orthogonal to other dearomative reactions.

Experimental Procedures

General Comments

All reactions were conducted in dried and deoxygenated solvents. Solvents for column chromatography were used without further purification. Commercially available starting materials were used as received and included all Ar—F except the pentafluorophenyl butyl ketone. Photocatalysts were synthesized according to literature procedures and structures were confirmed by 1H NMR spectroscopy. PC1 (Teegardin et al., 2018), PC4 (Singh et al., 2015), PC5 (Singh et al., 2015), PC7 (Singh et al., 2015), PC8 (Pearson et al., 2016), PC9 (Luo et al., 2016). PC2, PC3, and PC6 were purchased and used as received. Structural assignments were made with additional information from gCOSY, gHSQC, and gHMBC experiments.

Melting Points

Melting points were determined on a Stuart SMP10 melting point apparatus and are reported uncorrected.

NMR Analysis

NMR-spectra were recorded using a Bruker Avance 400 (400 MHz for ¹H, 101 MHz for ¹³C, 376 MHz for ¹⁹F) or a Bruker Neo 600 with BBO BBF-H-D-05 SmartProbe (599 MHz for ¹H, 564 MHz for ¹⁹F, 151 MHz for ¹³C), as noted. Chemical shifts are reported in ppm on the δ scale with residual solvent signal as internal standard. As abbreviations for the multiplicity were used: s=singlet, d=doublet, t=triplet, q=quartet, m=multiplet, pt=pseudo triplet, app=apparent.

Analysis of reaction mixtures was performed in undeuterated solvents with either a DMSO-d₆ or C₆D₆ capillary as reference. Spectra were analyzed using Topspin 4.0.6 or MestReNova 14.0.1-23559.

Thin Layer Chromatography

Thin layer chromatography was done on with silica gel pre-coated aluminum sheets (Machery-Nagel, silica gel 60 G/UV254, 0.2 mm) and for visualization UV-light (254 nm) and potassium permanganate stain.

High Resolution Mass Spectrometry

HRMS was measured on a ThermoFisher LTQ OrbitrapXL or an Agilent Q-TOF 6540 UHD.

X-Ray Analysis

X-Ray analysis was performed by the crystallography laboratory of the University of Regensburg. Structure solving was done by Florian Meurer.

General Procedure A (batch) for Formation of the 1,4-diene:

In a darkened lab space, into a new 25 mL test tube fitted with a stir bar was charged the fluoroarene (2.0 mmol) and a stock solution consisting of the Ar—H (3.0 equiv.), DIPEA (1.1 equiv.), Ir(dtbubpy)(dtbuppy)₂PF₆ (0.125 mol %), water (15 equiv.), a C6F6 internal standard (⅙ equiv.—testing showed no difference between reactions in which the standard was included and those in which it was not), and acetonitrile (0.1 M). The test tube was fitted with a rubber septum. This solution was chilled to 0° C. and then sparged with argon for 10 minutes at 0° C. The solution was attached to low, constant positive argon pressure and added to an irradiation bath at 460 nm and held at 0° C. until complete by NMR indication of complete consumption of SM. The reaction was then concentrated, extracted at least thrice with boiling hexanes, or until no remaining color was apparent in hexanes extracts. The pooled hexanes extracts were then concentrated and the resultant mixture heated in a 10 mL round bottomed flask at 90° C. under high vacuum for 1-2 hours. The resultant mixture was then dry loaded onto silica, and subjected to flash chromatography on a silica column with a hexanes and ethyl acetate mobile phase. The fractions containing the product were pooled and concentrated in a 20 mL scintillation vial. To the resultant oil was added a minimal amount of methanol, from which pure crystals formed upon repeated freezing of the solution in liquid nitrogen followed by vigorous shaking as the solution warmed.

General Procedure B (batch) for Formation of the 1,4-diene:

Perfluoroarene (12 mmol), aromatic trapping agent (31.2 mmol, 2.6 equiv.), and [Ir(dtbbpy)(dtbppy)₂]PF₆ (2 mg, 0.014 mol %) were dissolved in 120 mL of acetonitrile inside a Tauchschachtreactor. DIPEA (14.4 mmol, 2.52 mL, 1.2 equiv.) was added and the solution irradiated at 455 nm. After three days the solvent was removed under reduced pressure. The residue was dissolved in 100 mL boiling hexane and filtered to remove insoluble components. The solvent was removed under reduced pressure and most of the Ar—H coupling partner distilled off with Kugelrohr distillation (for methyl toluate and methyl acetophenone: 95° C., 1 mbar. 80-90% recovered). The residue was purified analogously to general procedure A.

General Procedure C (flow) for Formation of the 1,4-diene:

In a darkened lab space, same as above except in 250 mL PFA round bottom flask test tube fitted with a stir bar was charged the fluoroarene and a stock solution consisting of the Ar—H (3.0 equiv.), DIPEA (1.1 equiv.), Ir(dtbubpy)(dtbuppy)₂PF₆ (0.125 mol %), water (15 equiv.) and acetonitrile (0.1 M). The flask was fitted with a rubber septum. The solution was attached to low, constant positive argon pressure and circulated through PFA tubing via a peristaltic pump at 70 RPM, in which it was irradiated and held at room temp until complete by NMR indication of complete consumption of SM through analysis of aliquot. The products were isolated analogously to general procedure A.

General Procedure D (NMR scale)

Same as general procedure A, except in a clean, dry NMR tube. A sealed melting point capillary containing a deuterated solvent was included in the NMR tube. The tube was then chilled to 0° C. and degassed by sparging with argon through an 18-gauge stainless steel needle for 10 minutes. The NMR tube was fitted with a septum and sealed with parafilm.

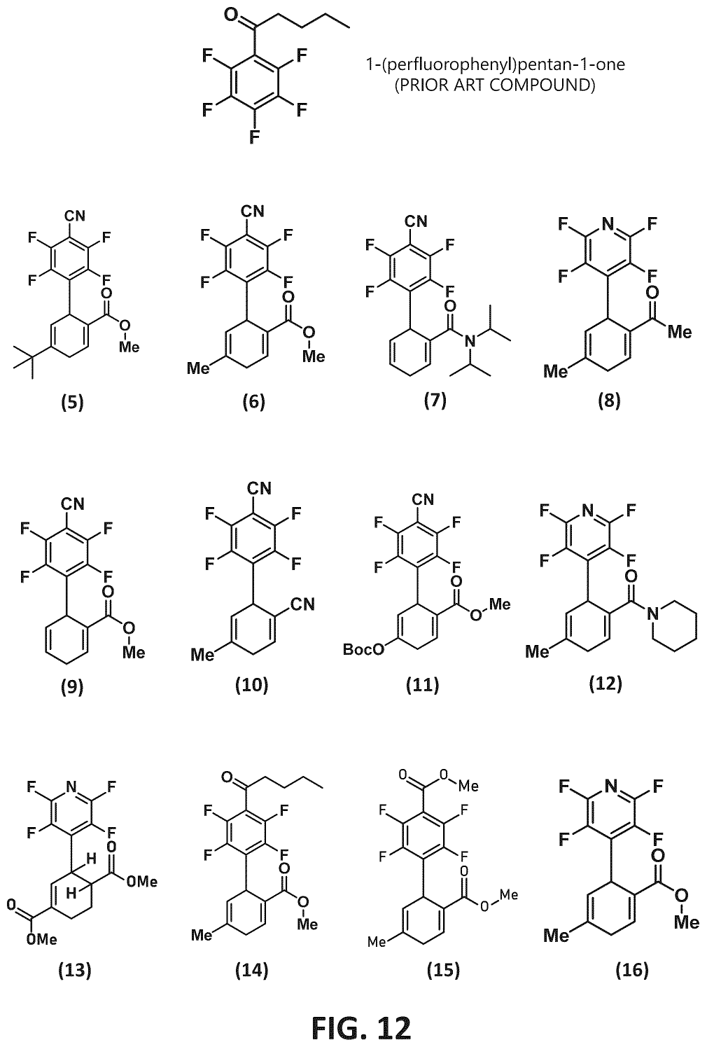

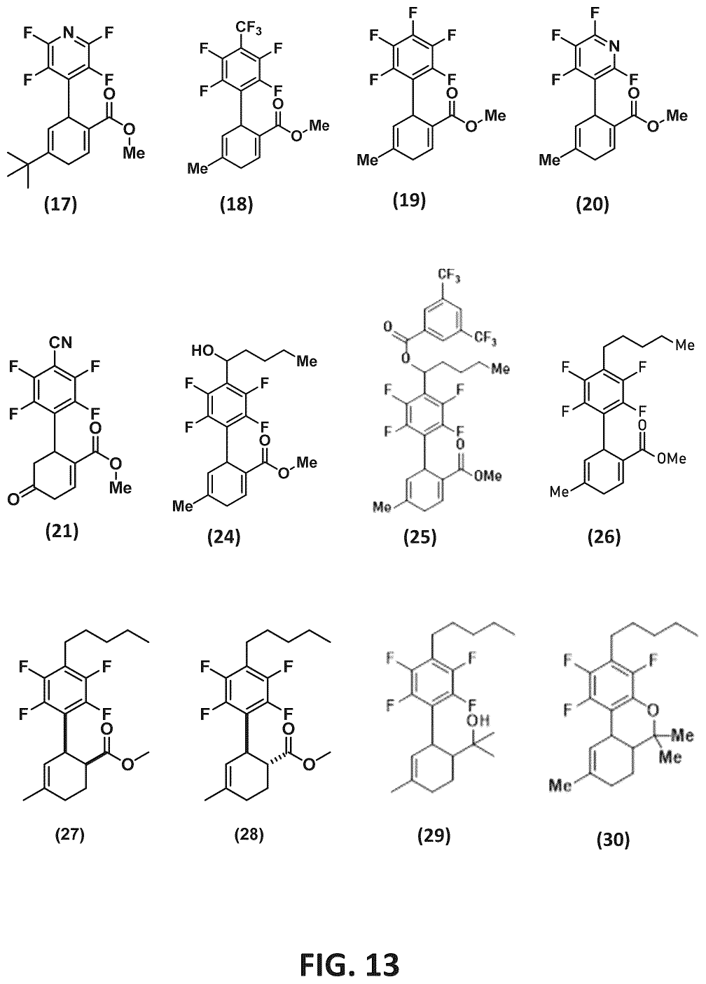

The chemical structures of the synthesized compounds listed below can be found in FIGS. 12-13.

Synthesis of 1-(perfluorophenyl)pentan-1-one

Literature known compound. A 1 M solution of bromopentafluorobenzene (6.17 g, 25 mmol) was prepared and slowly added to activated magnesium turnings (729 mg, 30 mmol, 1.2 equiv). First, enough of the solution was added to cover the Mg. A grain of iodine was added and the solution heated until the color of the iodine disappeared. The remaining solution is added dropwise while stirring to keep the reaction mixture slightly refluxing.

After all the bromopentafluorobenzene was added the mixture was stirred until it has cooled to room temperature. A solution of pentanal (2.37 g, 27.5 mmol, 1.1 equiv. based on bromopentafluorobenzene) in 20 mL THF was added to the Grignard solution. The reaction mixture was stirred overnight at rt, quenched with saturated NH₄Cl solution, extracted with ethyl acetate, dried over MgSO₄, and the solvent evaporated under reduced pressure. The crude alcohol was used for the Jones oxidation without further purification.

The crude secondary alcohol was dissolved in approximately 50 times its volume of acetone. The exact amount of solvent did not have any observable effect on the yield. A 2 M solution of CrO₃ in a 5:1 mixture (volume) of water and sulfuric acid was added in small portions while stirring until complete consumption of starting material. Reaction progress was monitored via TLC. After completion of the reaction isopropanol was added to decompose remaining Cr (VI). A small amount of water and ethyl acetate were added and the mixture extracted with ethyl acetate, washed with water, dried over MgSO₄, and the solvent removed under reduced pressure. The crude product was purified by vacuum distillation (100° C., 1 mbar) to give the product as a slightly yellow liquid. Isolated yield: 3.10 g, 11.9 mmol, 49% based on bromopentafluorobenzene. The spectra matched the literature values (Shen et al., 1988).

¹⁹F NMR (CDCl3 377 MHz): δ −141.42-−141.67 (m, 2 F), −150.06-−150.35 (m, 1 F), −160.06-−160.37 (m, 2 F). ¹H NMR (CDCl3, 400 MHz): δ 2.88 (t, 2H, J=7.48 Hz), 1.76-1.65 (m, 2 H), 1.46-1.34 (m, 2 H), 0.95 (t, 3H, J=7.3 Hz). ¹³C{¹H} NMR (CD₃CN, 101 MHz): δ 194.2, 144.0 (dm, J=252.86 Hz), 142.5 (dm, J=258.11 Hz), 137.6 (dm, J=255.97 Hz), 115.6-115.0 (m), 44.80, 25.55, 22.01, 13.50.

(5) Methyl 5-(tert-butyl)-4′-cyano-2′,3′,5′,6′-tetrafluoro-1,4-dihydro-[1,1′-biphenyl]-2-carboxylate. General Procedure A was followed. Colorless crystalline solid, MP 108-110° C. 19F NMR (CD₃CN, 376 MHz,): δ −136.63 (td, 2F, J=16.0, 7.1 Hz), −143.09 (td, 2F, J=16.0, 7.1 Hz). ¹H NMR (CD₃CN, 400 MHz): δ 7.28 (t, 1H, J=3.9 Hz), 5.60-5.36 (m, 1H), 4.89 (q, 1H, J=6.1 Hz), 3.62 (s, 3H), 3.17-3.00 (m, 2H), 1.08 (s, 9H). ¹³C{¹H} NMR (CD₃CN, 151 MHz): δ 165.6, 147.1 (ddt, J=257.6, 17.2, 4.0 Hz), 145.3 (d, J=248.2 Hz), 144.3, 140.6, 129.6 (t, J=14.7 Hz), 125.8, 114.2, 107.9 (t, J=3.8 Hz), 91.8 (tt, J=17.6, 3.1 Hz), 51.3, 34.7, 33.1 (t, J=2.1 Hz), 28.0, 26.4. HRMS (ESI/ion trap) m/z: [M−H+]-Calcd for C₁₉H₁₆F₄NO-366.1123; Found 366.1137. NMR yield: 32.9%. Isolated yield: 151.0 mg, 20.5%.

(6) Methyl 4′-cyano-2′,3′,5′,6′-tetrafluoro-5-methyl-1,4-dihydro-[1,1′-biphenyl]-2-carboxylate. General procedure C was followed. Colorless crystalline solid, MP 91-95° C. ¹⁹F NMR (CDCl₃, 376 MHz): δ −133.47 (td, 2F, J=16.6, 7.4 Hz), −141.19 (td, 2F, J=15.9, 6.8 Hz). ¹H NMR (CDCl₃, 400 MHz): δ 7.25 (t, 1H, J=3.8 Hz), 5.39-5.22 (m, 1H), 4.83 (q, 1H, J=5.6 Hz), 3.67 (s, 3H), 2.98 (dd, 1H, J=23.8, 7.9 Hz), 2.85 (dt, 1H, J=23.9, 5.5 Hz), 1.76 (s, 3H). ¹³C{¹H} NMR (CDCl₃, 101 MHz): δ 166.0, 147.1 (ddt, J=261.5, 16.6, 3.7 Hz), 145.2 (app-dm, J=250.4 Hz), 140.3, 133.2, 129.5 (t, J=14.3 Hz), 126.4, 117.5, 107.9 (t, J=3.8 Hz), 92.3, 52.0, 33.2 (t, J=2.1 Hz), 32.1, 22.7. HRMS (ESI/ion trap) m/z: [M−H⁺]⁻ Calcd for C₁₆H₁₀F₄NO₂⁻ 324.0653; Found 324.0665. Isolated yield: 552.9 mg, 17.0%.

(7) 4′-Cyano-2′,3′,5′,6′-tetrafluoro-N,N-diisopropyl-1,4-dihydro-[1,1′-biphenyl]-2-carboxamide. General Procedure A was followed, except the Ar—H was not removed by heating under high vac, but rather separated by column chromatography, and the material was found to crystallize only from pentane. 40 g silica column, material eluted with large amount of benzamide Ar—H. Second 24 g silica column was run on these combined fractions, which provided a peak which contained the intended product and the biaryl. Recrystallization from pentane. Colorless crystalline solid, MP 165-167° C. ¹⁹F NMR (CDCl₃, 376 MHz) δ −135.27-−135.57 (m, 2F), −142.00-−142.19 (m, 2F). ¹H NMR (CD₃CN, 400 MHz): δ 6.03-5.91 (m, 2H), 5.67 (ddt, 1H, J=10.0, 3.9, 2.1 Hz), 4.90-4.80 (m, 1H), 3.75 (s, 3H), 3.04-2.74 (m, 2H), 1.21 (d, 6H, J=6.7 Hz), 1.01 (s, 6H). ¹³C{¹H} NMR (CD₃CN, 101 MHz): δ 169.4, 147.6 (ddt, 4.0 Hz, J=258.7, 16.9), 145.9 (app. dm, J=248.4 Hz), 131.7, 130.7, 129.6, 128.7 (t, J=15.0 Hz), 126.6, 125.1, 123.0, 108.3 (t, J=3.8 Hz), 93.1 (tt, J=17.6, 3.0 Hz), 33.6 (p, J=1.5 Hz), 26.1, 20.5, 20.2. HRMS (ESI/ion trap) m/z: [M−H⁺]⁻ Calcd for C₂₀H₁₉F₄N₂O⁻ 379.1439; Found 379.1458. NMR yield: 25.0%. Isolated yield: 19.7 mg, 2.6%.

(8) 1-(4-Methyl-6-(perfluoropyridin-4-yl)cyclohexa-1,4-dien-1-yl)ethan-1-one Synthesized according to general procedure 3 from pentafluoropyridine and p-methyl acetophenone. Purification via column chromatography (5% EA in PE) yielded mostly clean product, contaminated with rearomatized product as a viscous yellow liquid which solidified within three days. Recrystallization from n-hexane or MeOH yielded the clean product. Colorless crystalline solid. MP 57-58° C. The structure of this compound could be definitely determined by single crystal X-ray diffractometry. TLC: R_{f}=0.35 (hexanes/ethyl acetate 9:1). ¹⁹F-NMR (CDCl₃, 377 MHz): δ=−92.86-−93.10 (m, 2F), −146.26-−146.51 (m, 2F). ¹H-NMR (CDCl₃, 400 MHz): δ 7.19-7.14 (m, 1H), 5.37-5.29 (m, 1H), 4.86-4.77 (m, 1H), 3.14-2.99 (m, 1H), 2.99-2.85 (m, 1H), 2.29 (s, 3H), 1.76 (s, 3H). ¹³C{¹H} NMR (CDCl₃, 101 MHz): δ 197.1, 144.8-144.4 (m), 142.3-141.9 (m), 141.9-141.5 (m), 141.1, 139.4-138.9 (m), 136.7-136.2 (m), 135.7, 132.6, 117.6, 32.3, 32.2, 25.0, 22.4. HRMS (+APCI): Calcd for [C₁₄H₁₁F₄O+NH₄⁺]⁺: 303.1115, found: 303.1133. Isolated yield: 765 mg, 22%.

(9) Methyl 4′-cyano-2′,3′,5′,6′-tetrafluoro-1,4-dihydro-[1,1′-biphenyl]-2-carboxylate. General Procedure A was followed. 4 g silica column. Colorless crystalline solid, MP 147-149° C. ¹⁹F NMR (CD₃CN 376 MHz): δ −136.45 (td, J=16.0, 7.0 Hz), −143.01 (app. td, J=16.1, 7.0 Hz). ¹H NMR (CD₃CN, 400 MHz): δ 7.25 (t, 1H, J=4.0 Hz), 6.18-5.86 (m, 1H), 5.76-5.52 (m, 1H), 5.03-4.73 (m, 1H), 3.63 (s, 3H), 3.17-2.90 (m, 2H). ¹³C{¹H} NMR (CD₃CN, 101 MHz): δ 166.6, 148.2 (ddt, J=257.8, 17.1, 3.9 Hz), 146.2 (d, J=247.6 Hz), 140.9 (t, J=1.4 Hz), 130.1 (t, J=14.7 Hz), 127.4, 126.3, 123.6, 108.9 (t, J=3.8 Hz), 93.1 (tt, J=17.5, 2.6 Hz), 52.4, 32.9 (p, J=2.1 Hz), 27.8. HRMS (ESI/ion trap) m/z: [M−H⁺]⁻ Calcd for C₁₅H₈F₄NO₂⁻ 310.0497; Found 310.0509. NMR yield: 25.0%. Isolated yield: 98.5 mg, 15.8%.

(10) 2′,3′,5′,6′-Tetrafluoro-5-methyl-1,4-dihydro-[1,1′-biphenyl]-2,4′-dicarbonitrile. General Procedure A was followed, except that the material was repeated recrystallized either from hexanes or methanol. Colorless crystalline solid, MP 117-120° C. ¹⁹F NMR (CDCl₃, 376 MHz): δ −131.79 (td, 2F, J=16.8, 7.3 Hz), −140.34 (td, 2F, J=16.6, 6.5 Hz). ¹H NMR (CDCl₃, 400 MHz): δ 6.87 (td, 6H, J=3.7, 1.6 Hz), 5.44-5.19 (m, 6H), 4.85-4.49 (m, 6H), 3.13-2.73 (m, 12H), 1.78 (s, 20H), 1.25 (s, 1H). ¹³C{¹H} NMR (CDCl₃, 151 MHz): δ 147.4 (ddt, J=259.1, 16.8, 3.9 Hz), 145.2 (ddt, J=250.0, 11.7, 5.2 Hz), 145.0, 133.2, 131.8, 126.1 (t, J=14.4 Hz), 115.4, 108.7, 107.7 (t, J=3.9 Hz), 93.6 (tt, J=17.4, 2.6 Hz), 34.2, 30.9, 21.7. HRMS (ESI/ion trap) m/z: [M−H⁺]⁻ Calcd for C₁₅H₇F₄N₂⁻ 291.0551; Found 291.0552. NMR yield: 16.4%. Isolated yield: 30.0 mg, 9.1%.

(11) Methyl 5-((tert-butoxycarbonyl)oxy)-4′-cyano-2′,3′,5′,6′-tetrafluoro-1,4-dihydro-[1,1′-biphenyl]-2-carboxylate. General Procedure A was followed, but with only 1 equivalent water, and the Ar—H was not removed by heating under high vac, but rather separated by column chromatography. 40 g silica column. Colorless crystalline solid, MP 104-105° C. ¹⁹F NMR (CDCl₃, 376 MHz): δ −132.84 (td, 2F, J=16.6, 7.3 Hz), −140.61 (td, 2F, J=16.5, 15.8, 6.7 Hz). ¹H NMR (CDCl₃, 400 MHz): δ 7.13 (ddd, 1H, J=4.4, 3.0, 1.0 Hz), 5.43 (dd, J=4.3, 2.0 Hz, 1H), 4.98 (q, J=6.1 Hz, 1H), 3.61 (s, 3H), 3.23 (ddt, 1H, J=23.4, 7.5, 2.7 Hz), 3.06 (ddd, 1H, J=23.4, 6.4, 4.6 Hz), 1.43 (s, 9H). ¹³C{¹H} NMR (CDCl₃, 101 MHz): δ 165.7, 151.0, 147.5, 147.5 (ddt, J=262.2, 16.6, 3.1 Hz), 145.6 (d, J=250.0 Hz), 139.0, 128.3 (t, J=14.0 Hz), 126.6, 110.3, 108.0 (t, J=3.6 Hz), 84.3, 52.6, 33.5, 31.4, 29.2, 28.1. HRMS (ESI/ion trap) m/z: [M−H]⁻ Calcd for C₂₀H₁₆F₄NO₅⁻ 426.0970; Found 426.0995. Isolated yield: 118.6 mg, 13.8%.

(12) (4-Methyl-6-(perfluoropyridin-4-yl)cyclohexa-1,4-dien-1-yl) (piperidin-1-yl)methanone. General Procedure B was followed, at a 12 mmol scale, with 2.6 equiv. ArH, 10 equiv. water. The Ar—H was not removed by heating under high vac, but rather separated by column chromatography. Recrystallized from n-hexane. Colorless crystalline solid. ¹⁹F NMR (CDCl₃, 376 MHz): δ −92.00-−92.38 (m, 2F), −145.53-−145.89 (m, 2F). ¹H NMR (CDCl₃, 400 MHz): δ 6.08 (ddd, 1H, J=4.2, 2.9, 1.7 Hz), 5.32 (td, 1H, J=3.1, 1.6 Hz), 4.93 (q, 1H, J=6.4 Hz), 3.65-3.22 (m, 3H), 2.89 (dd, 1H, J=23.3, 6.8 Hz), 2.75 (ddd, 1H, J=22.9, 7.3, 4.0 Hz), 1.75 (s, 3H), 1.59 (p, 2H, J=5.8 Hz), 1.53-1.18 (m, 5H). ¹³C{¹H} NMR (CDCl₃, 101 MHz): δ 168.6, 145.0-141.9 (m), 140.6 (dd, J=260.6, 20.8 Hz), 135.4 (t, J=13.3 Hz), 133.5, 129.0, 128.3, 117.0, 34.4, 31.6, 30.9, 26.2, 24.5, 22.7, 14.1. HRMS (+ESI) m/z: [M+H]⁺ Calcd for C₁₈H₁₉F₄N₂O⁺ 355.1428; Found 355.1423. Isolated yield: 220.0 mg, 6.2%. The reaction was repeated at a later stage with organic photocatalyst 4CzIPN (PC9). Crude NMR sowed similar results. Isolated yield was 9.9% due slower gradient during column chromatography (0→5% EtOAc in hexanes) and better separation.

(13) Dimethyl 3-(perfluoropyridin-4-yl)cyclohex-1-ene-1,4-dicarboxylate

General Procedure B was followed. The Ar—H was not removed by heating under high vac, but rather separated by column chromatography. MP 93-94° C. ¹⁹F NMR (CDCl₃, 376 MHz): δ −90.76-−91.23 (m, 2F), −142.62-−143.66 (m, 2F). ¹H NMR (CDCl₃, 400 MHz): δ 6.84-6.45 (m, 1H), 4.35 (d, 1H, J=10.5 Hz), 3.74 (s, 3H), 3.63 (s, 3H), 2.95 (ddd, 1H, J=12.5, 10.4, 2.8 Hz), 2.77-2.56 (m, 1H), 2.54-2.22 (m, 2H), 1.99-1.65 (m, 1H). ¹³C{¹H} NMR (CDCl₃, 101 MHz) δ 173.5, 166.6, 143.7 (dddd, J=246.0, 16.5, 13.0, 3.1 Hz), 140.7 (d, J=259.5), 134.8, 134.7 (tt, J=14.3, 2.4 Hz), 132.1, 52.4, 52.1, 43.2 (t, J=2.2 Hz), 35.8 (t, J=2.0 Hz), 26.0, 23.9. HRMS (ESI/ion trap) m/z: [M+H]⁺ Calcd for C₁₅H₁₄F₄NO₄⁺ 348.0853; Found 348.0856. Isolated yield, 200 mg, 575.93 umol, 4.8%.

(14) Methyl 2′,3′,5′,6′-tetrafluoro-5-methyl-4′-pentanoyl-1,4-dihydro-[1,1′-biphenyl]-2-carboxylate General procedure B was followed from 1-(perfluorophenyl)pentan-1-one and methyl p-toluate. Purification via column chromatography (5% EA in PE) yielded the product contaminated with variable amounts of rearomatized product as a yellow, viscous liquid. Recrystallization from MeOH yielded the product as colorless needles. MP 66.8° C. TLC: R_{f}=0.50 (hexanes/ethyl acetate 9:1). ¹⁹F-NMR (CDCl₃, 376 MHz): δ [ppm]=−143.85-−14.17 (m, 4F) ¹H-NMR (CDCl₃, 400 MHz): δ 7.24-7.17 (m, 1H), 5.42-5.21 (m, 1H), 4.78 (q, 1H, J=6.2, 5.8 Hz), 3.65 (s, 3H), 3.11-2.70 (m, 4H), 1.73 (t, 3H, J=1.4 Hz), 1.67 (p, J=7.4 Hz, 2H), 1.36 (h, J=7.4 Hz, 2H), 0.91 (t, J=7.3 Hz, 3H). ¹³C{¹H} NMR (CDCl₃, 101 MHz): 5195.8, 166.1, 145.2 (ddt, J=253.8, 16.3, 5.4 Hz), 143.5 (dddd, J=254.4, 17.6, 6.5, 5.0 Hz), 139.7, 132.2, 127.0, 124.6 (t, J=14.0 Hz), 118.3, 118.1 (t, J=17.8 Hz), 51.8, 44.8, 32.6 (p, J=1.9 Hz), 32.0, 25.7, 22.6, 22.2, 13.8. HRMS (EI): exact mass calc. for C₂₀H₂₀F₄O₃: m/z=384.1349, found: 384.1337 [M]⁺. Isolated yield 947 mg, 2.46 mmol, 21%.

(15) Dimethyl 2′,3′,5′,6′-tetrafluoro-5-methyl-1,4-dihydro-[1,1′-biphenyl]-2,4′-dicarboxylate General Procedure A was followed. Product eluted at 5% EtOAc in Hexanes after 38 CV in 24 g column. Colorless crystalline solid. MP 91-95° C. ¹⁹F NMR (CDCl₃, 564 MHz): δ −139.20 (dd, J=22.2, 12.6 Hz, 2F (biaryl impurity)), −139.46 (dd, J=22.6, 13.0 Hz, 2F (biaryl impurity)), −140.33-−140.90 (m, 2F), −142.99-−143.96 (m, 2F). ¹H NMR (CDCl₃, 599 MHz) δ 7.25-7.16 (m, 1H), 5.39-5.25 (m, 1H), 4.81 (q, 1H, J=5.5, 5.0 Hz), 3.95 (s, 3H), 3.66 (s, 2H), 2.97 (ddt, 1H, J=24.8, 6.5, 2.4 Hz), 2.83 (dt, 1H, J=23.8, 5.5 Hz), 1.74 (s, 3H). ¹³C{¹H} NMR (CDCl₃, 151 MHz): δ 166.2, 160.6, 145.3 (ddt, J=249.6, 14.3, 4.7 Hz), 144.7 (ddt, J=256.7, 15.9, 4.4 Hz), 139.8, 132.4, 126.9, 125.7 (t, J=14.7 Hz), 118.2, 110.5 (t, J=15.8 Hz), 53.3, 51.9, 32.7, 32.1, 22.6. HRMS (ESI/ion trap) m/z: [M+Na⁺]⁺ Calcd for C₁₇H₁₃F₄O₄ Na 381.0726; Found 381.0707. Isolated yield: 99.6 mg, 13.8%.

(16) Methyl 4-methyl-6-(perfluoropyridin-4-yl)cyclohexa-1,4-diene-1-carboxylate General Procedure A was followed. Separation via 4 g chromatography column. Colorless crystalline solid. MP 93-96° C. ¹⁹F NMR (CD₃CN, 564 MHz) δ −94.96-−95.25 (m, 2F), −146.97-−147.15 (m, 2F). ¹H NMR (CD₃CN, 599 MHz): δ 7.38-7.13 (m, 1H), 5.45-5.34 (m, 1H), 4.93-4.82 (m, 1H), 3.63 (s, 3H), 3.02 (app. dd, 1H, J=24.3, 7.2, Hz), 2.92 (dddt, 1H, J=24.1, 6.5, 4.5, 0.9 Hz), 1.79-1.76 (m, 3H). ¹³C{¹H} NMR (CD₃CN, 151 MHz): 166.6, 144.3 (apparent d, J=241.7 Hz), 141.8 (apparent d, J=256.9 Hz), 137.5 (tt, J=13.3, 2.2 Hz), 134.3, 127.1, 117.7, 52.3, 34.1 (t, J=1.9 Hz), 32.4, 30.9, 22.6. HRMS (ESI/ion trap) m/z: [M−H⁺]⁻ Calcd for C₁₄H₁₀F₄NO₂⁻ 300.0653; Found 300.0658. NMR yield: 13.2%. Isolated yield: 69.1 mg, 11.4%.

(17) Methyl 4-(tert-butyl)-6-(perfluoropyridin-4-yl)cyclohexa-1,4-diene-1-carboxylate General Procedure A was followed. Colorless crystalline solid. MP 59-60° C. ¹⁹F NMR (CDCl₃, 376 MHz) δ −91.85-−92.17 (m, 2F), −145.34-−145.88 (m, 2F). ¹H NMR (CDCl₃, 400 MHz) δ 7.30 (td, 1H, J=3.9, 1.1 Hz), 5.40 (dt, 1H, J=4.3, 1.5 Hz), 4.89 (q, 1H, J=5.9 Hz), 3.67 (s, 3H), 3.05 (ddd, J=6.7, 4.0, 1.5 Hz, 2H), 1.07 (s, 9H). ¹³C{¹H} NMR (CDCl₃, 101 MHz): δ 165.9, 144.9, 143.6 (d, J=248.4 Hz), 141.1, 140.8 (d, J=273.3 Hz), 136.3 (t, J=13.1 Hz), 125.8, 114.1, 52.0, 35.2, 33.5 (t, J=1.8 Hz), 28.8, 26.9 (t, J=1.6 Hz). HRMS (ESI/ion trap) m/z: [M−H⁺]⁻ Calcd for C₁₇H₁₆F₄NO₂⁻ 342.1123; Found 342.1143. NMR yield: 16.7%. Isolated yield: 99.5 mg, 14.5%.

(18) Methyl 2′,3′,5′,6′-tetrafluoro-5-methyl-4′-(trifluoromethyl)-1,4-dihydro-[1,1′-biphenyl]-2-carboxylate General Procedure A was followed, except the product was recrystallized from isopropanol. Colorless crystalline solid. MP 104-105° C. ¹⁹F NMR (CD₃CN, 564 MHz): δ −57.07 (t, 3F, J=21.3 Hz), −144.10-−144.22 (m, 2F), −144.22-−144.46 (m, 2F). ¹H NMR (CD₃CN, 400 MHz): δ 7.24-7.17 (m, 1H), 5.41-5.34 (m, 1H), 4.83 (q, 1H, J=5.5 Hz), 3.6 (s, 3H), 2.99 (ddt, 1H, J=24.2, 6.6, 2.5 Hz), 2.88 (ddd, 1H, J=24.1, 6.5, 4.6 Hz), 1.74 (s, 3H). ¹³C{¹H} NMR (CD₃CN, 151 MHz): δ 166.6, 146.5 (d, J=246.3 Hz), 145.0 (d, J=235.4 Hz), 140.7, 133.8, 128.2 (t, J=14.7 Hz), 127.4, 122.2 (q, J=273.3 Hz), 108.5-107.4 (m), 52.2, 33.6 (t, J=2.1 Hz), 32.4, 22.5. HRMS (ESI/ion trap) m/z: [M−H⁺]⁻ Calcd for C₁₆H₁₀F₇O₂⁻ 367.0575; Found 367.0587. NMR yield: 29.3%. Isolated yield: 84.0 mg, 11.4%.

(19) Methyl 2′,3′,4′,5′,6′-pentafluoro-5-methyl-1,4-dihydro-[1,1′-biphenyl]-2-carboxylate General Procedure A was followed from bromopentafluorobenzene. Colorless oily semisolid. Partial isolation was achieved via silica gel chromatography, 4 g hexanes/ethyl acetate. ¹⁹F NMR (CD₃CN, 376 MHz): δ −146.20 (dd, 2F, J=21.2, 7.8 Hz), −160.17 (td, 1F, J=20.2, 1.1 Hz), −165.56-−165.83 (m, 2F). ¹H NMR (CD₃CN, 400 MHz): δ 7.22-7.12 (m, 1H), 5.41-5.34 (m, 1H), 4.74 (q, 1H, J=6.1 Hz), 3.03-2.90 (m, 1H), 2.92-2.79 (m, 1H), 1.73 (s, 3H). ¹³C{¹H} NMR (CD₃CN, 151 MHz): δ 165.8, 145.4 (d, J=245.3 Hz), 139.8 (d, J=247.4 Hz), 139.3, 137.5 (d, J=247.5 Hz), 132.2, 129.2, 127.0, 118.2, 51.2, 31.9, 31.4, 21.5. HRMS (ESI/ion trap) m/z: [M−H⁺]⁻ Calcd for C₁₅H₁₀F₅O₂⁻ 317.0606; Found 317.0628. NMR yield, 18.1%.

(20) Methyl 4-methyl-6-(perfluoropyridin-3-yl)cyclohexa-1,4-diene-1-carboxylate General Procedure A was followed with 3-chloro teterafluoropyridine. Partial resolution of atropisomers was achieved through repeated recrystallizations. HRMS (ESI/ion trap) m/z: [M−H⁺]⁻ Calcd for C₁₄H₁₀F₄NO₂⁻ 300.0653; Found 300.0668. NMR yield: 21.3%. 2.4:1 dr.

Atropisomer 1 (Major):

¹⁹F NMR (CD₃CN, 376 MHz): δ −73.99, −89.41 (ddd, J=22.2, 18.8, 14.6 Hz, 1F), −118.89 (q, J=17.2 Hz, 1F), −168.50 (ddd, J=24.0, 21.8, 18.4 Hz, 1F). ¹H NMR (CD₃CN, 400 MHz): δ 6.77 (m, 1H), 5.75 (m, 1H), 4.47 (q, 1H, J=6.5 Hz), 3.73 (s, 3H), 3.00 (ddp, 2H, J=7.6, 3.9, 2.0 Hz), 1.67-1.56 (m, 3H). ¹³C{¹H} NMR (CD₃CN, 151 MHz): δ 167.6, 159.7 (d, J=261.0 Hz), 154.1 (d, J=242.3 Hz), 149.1 (d, J=240.2 Hz), 134.4 (d, J=256.4 Hz),134.5, 130.4, 129.1, 122.4, 112.2, 52.3, 37.1, 26.8, 20.8.

Atropisomer 2 (Minor):

¹⁹F NMR (CD₃CN, 376 MHz): δ −74.63 (dt, J=21.0, 14.3 Hz), −90.73 (ddd, J=21.9, 18.2, 14.8 Hz, 1F), −119.82 (tdd, 1F, J=18.2, 14.8, 1.2 Hz), −169.33 (ddd, 1F, J=23.9, 22.0, 18.4 Hz). ¹H NMR (CD₃CN, 400 MHz): δ 7.22 (m, 1H), 5.40 (m, 1H), 4.74-4.60 (m, 1H), 3.62 (s, 3H), 3.07-2.94 (m, 1H), 2.94-2.79 (m, 1H), 1.77 (s, 3H). ¹³C{¹H} NMR (CD₃CN, 151 MHz): δ 166.7, 159.3 (d, J=262.2 Hz), 153.8 (d, J=237.5 Hz), 148.4 (d, J=244.8 Hz), 134.0 (d, J=267.7 Hz), 133.5, 127.5, 112.0, 118.6, 52.2, 140.5, 32.6, 32.3, 22.5.

(21) Methyl 4′-cyano-2′,3′,5′,6′-tetrafluoro-5-oxo-1,4,5,6-tetra hydro-[1,1′-biphenyl]-2-carboxylate

Methyl 5-((tert-butoxycarbonyl)oxy)-4′-cyano-2′,3′,5′,6′-tetrafluoro-1,4-dihydro-[1,1′-biphenyl]-2-carboxylate (11) 5.0 mg (0.0117 mmol) was dissolved in DCM (0.02 M) in an NMR tube. Added 10 equivalents trifluoroacetic acid (TFA) and sonicated 2 hours. An additional 50 equivalents TFA was added and the mixture sonicated 2 hours. An additional 50 equivalents TFA was added and the mixture sonicated 2 hours, after which TLC indicated complete consumption of (11). ¹⁹F NMR (CDCl₃, 376 MHz): δ −131.70-−132.11 (m, 2F), −138.94-−139.44 (m, 2F). ¹H NMR (CDCl₃, 400 MHz): δ 7.31 (dd, 1H, J=4.4, 3.4 Hz), 4.88 (d, 1H, J=8.5 Hz), 3.72 (s, 3H), 3.24 (s, 2H), 2.96 (dd, 1H, J=15.7, 8.6 Hz), 2.63 (dd, 1H, J=15.6, 3.1 Hz). HRMS (ESI/ion trap) m/z: [M−H+]− Calcd for C₁₅H₈F₄NO₃⁻ 326.0446; Found 326.0453. NMR yield: 94%.

(24) Methyl 2′,3′,5′,6′-tetrafluoro-4′-(1-hydroxypentyl)-5-methyl-1,4-dihydro-[1,1′- biphenyl]-2-carboxylate

Reaction derived from literature procedure⁷⁶ for deoxygenation of aromatic ketones. Compound (14) (77 mg, 200 μmol) was dissolved in 1 mL DCE. Anhydrous ZnI₂ (96 mg, 300 μmol, 1.5 equiv.) and NaBH₃CN (94 mg, 7.5 equiv.) were added and the dispersion heated to 80° C. in an oil bath for 6 h. After cooling to room temperature 2 mL 2M HCl was added dropwise until excess NaBH₃CN was decomposed. The solution was extracted with DCM, the organic layer dried with Na₂SO₄ and the solvent removed under reduced pressure. Purification via column chromatography (10% EA in PE) yielded the product as a slightly yellow viscous liquid. Solidified from CHCl₃ as a white, waxy solid.

TLC: R_{f}=0.30 (hexanes/ethyl acetate 9:1). ¹⁹F-NMR (CDCl₃, 376 MHz): δ [ppm]=−145.70 (dd, 2F, J=21.51, 12.24 Hz), −146.51 (dd, 2F, J=21.50, 12.24 Hz). ¹H-NMR (CDCl₃, 400 MHz): δ [ppm]=7.13 (d, J=2.81 Hz, 1H), 5.27 (s, 1 H), 5.00-4.89 (m, 1 H), 4.75-4.67 (m, 1H), 3.58 (s, 3H), 2.98-2.67 (m, 2H), 2.20-2.07 (m, 1H), 1.96-1.86 (m, 1 H), 1.80-1.70 (m, 1H), 1.67 (s, 3H), 1.44-1.11 (m, 5H), 0.83 (t, 3H). ¹³C{¹H} NMR (CDCl₃, 101 MHz): δ 166.3, 145.1 (d, J=245.8 Hz), 144.4 (d, J=245.0 Hz), 139.4, 131.6, 127.3, 120.9 (t, J=14.7 Hz), 120.3 (t, J=15.2 Hz), 118.8, 66.7, 51.7, 36.7, 32.3 (t, J=2.2 Hz), 32.0, 31.6, 28.0, 22.7, 22.5, 22.4, 14.1, 13.9. HRMS (EI): exact mass calc. for C₂₀H₂₂F₄O₃: m/z=386.1500, found: 386.1498 [M]+. Isolated yield: 75 mg, 194 μmol, 97%.

(25) Methyl 4′-(1-((3,5-bis(trifluoromethyl)benzoyl)oxy)pentyl)-2′,3′,5′,6′-tetrafluoro-5-methyl-1,4-dihydro-[1,1′-biphenyl]-2-carboxylate

According to a procedure derived from Reiser and coworkers,⁷⁵ compound (24) (900 mg, 2.33 mmol) was dissolved in 24 mL DCM and cooled in an ice bath. 3,5-bis(trifluoromethyl)benzoyl chloride (460 μL, 1.1 equiv.) was added dropwise. The solution was warmed to room temperature and the solvent removed under reduced pressure. The residue was extracted with hot petroleum ether, most of the solvent was removed under reduced pressure and the product purified via column chromatography (0-10% EA in PE) to yield the product as a colorless viscous liquid as a mixture of diastereomers. The diastereomers are not distinguishable in the ¹⁹F and ¹H NMR but most of the ¹³C resonances appear as slightly split signals.

TLC: R_{f}=0.70 (hexanes/ethyl acetate 9:1). ¹⁹F NMR (CDCl₃, 376 MHz): δ −63.62 (s, 6F), −144.34-−144.57 (m, 2F), −145.02 (dd, J=21.3, 12.2 Hz, 2F). ¹H NMR (CDCl₃, 400 MHz): δ [ppm]=8.49 (s, 2H), 8.07 (s, 1H), 7.22-7.18 (m, 1H), 6.31 (t, J=7.43 Hz, 1H), 5.37-5.31 (m, 1H), 4.83-4.74 (m, 1H), 3.67-3.63 (2s, overlapping, 3H), 3.04-2.90 (m, 1H), 2.81 (dt, 1H, J=23.74, 5.26 Hz), 2.33-220 (m, 1H), 2.11-2.00 (m, 1H), 1.77-1.70 (m, 3H), 1.51-1.25 (m, 4H+2H impurities), 0.92 (t, J=7.09 Hz, 3H). ¹³C{¹H} NMR (CDCl₃, 101 MHz): δ 166.3 (d, J=4.8 Hz), 163.3 (d, J=3.5 Hz), 145.3 (d, J=246.5 Hz), 144.7 (d, J=246.9 Hz), 139.6 (d, J=8.2 Hz), 132.5 (q, J=34.0 Hz), 132.2 (d, J=2.2 Hz), 130.0 (d, J=3.9 Hz), 127.2, 127.0-126.4 (m), 123.0 (q, J=273.1 Hz), 122.4 (td, J=14.7, 3.0 Hz), 118.7 (d, J=2.2 Hz), 115.9 (t, J=14.7 Hz), 69.4, 51.8 (d, J=2.6 Hz), 33.4, 32.5, 32.1, 27.8, 22.7, 22.6, 22.3, 13.9. Isolated yield: 1.30 g, 2.08 mmol, 89%.

(26) Methyl 2′,3′,5′,6′-tetrafluoro-5-methyl-4′-pentyl-1,4-dihydro-[1,1′-biphenyl]-2-carboxylate

According to a procedure derived from Reiser and coworkers,⁷⁵ (25) (376 mg, 600 μmol), 10 mg [Ir(dtbbpy)(dtbppy)₂]PF₆ and DIPEA (210 μL, 2 equiv.) were dissolved in a mixture of 15 mL acetonitrile and 1 mL of water. The solution was degassed and irradiated for 3 h at 455 nm while heating to 45° C. in an oil bath. After cooling to room temperature 20 mL petroleum ether was added, the solution washed with concentrated K₂CO₃ solution and water, dried over Na₂SO₄, the solvent removed under reduced pressure and the residue purified via column chromatography (5% EA in PE) yielded the product contaminated with small amounts of rearomatized product as a slightly yellow viscous liquid. Recrystallization from MeOH yielded the product as colorless needles. MP 54-55° C. TLC: R_{f}=0.70 (hexanes/ethyl acetate 9:1). ¹⁹F NMR (CDCl₃, 376 MHz): δ −146.66-−147.14 (m, 4F). ¹H NMR (CDCl₃, 400 MHz): δ 7.21-7.16 (m, 1H), 5.40-5.31 (m, 1H), 4.82-4.71 (m, 1H), 3.65 (s, 3H), 3.03-2.91 (m, 1H), 2.80 (dpt, 1H, J=23.7, 5.4 Hz), 2.65 (t, J=7.7, 2H), 1.74 (s, 3H), 1.62-1.52 (m, 2H), 1.26-1.28 (m, 4H), 0.89 (t, J=6.9 Hz). ¹³C{¹H} NMR (CDCl₃, 101 MHz): δ 166.2, 146.3-145.8 (m), 143.9-143.3 (m), 139.0, 131.2, 127.5, 119.1, 119.02-118.40 (m), 51.5, 32.1, 31.9, 31.3, 28.9, 22.6, 22.4, 22.3, 13.8. HRMS (EI): exact mass calc. for C₂₀H₂₂F₄O₂: m/z=370.1556, found: 370.1545 [M]⁺. Isolated yield: 193 mg, 521 μmol, 86%.

(27) & (28) Methyl 2′,3′,5′,6′-tetrafluoro-5-methyl-4′-pentyl-1,2,3,4-tetrahydro-[1,1′-biphenyl]-2-carboxylate Diene (26) (800 μmol) and PMHS (101 mg, 1.68 mmol, 2.1 equiv.) are put into a crimp vial under nitrogen. A solution of tris(pentafluorophenyl)borane (4 mg, 8 μmol, 1 mol %) in 4 mL DCM is added. The solution is stirred overnight at room temperature and stirred for another 24 h with saturated NH₄F solution or 2 h with TBAF·3H₂O in DCM. Shorter times for quenching lead to incomplete cleavage of the silyl ethers formed and diminish the yield. The solution is extracted with DCM and column chromatography (5% EA in PE) yields the product. The products are obtained as mixtures of cis and trans isomer which are not separable by means of column chromatography. Column chromatography (5% EA in PE) yielded the product as a colorless, viscous liquid. Different reactions gave the product in varying dr of 4-6 in favor of the cis diastereomer. Isolated yield: 26 mg, 70 μmol, 37% .

(27) (cis) Methyl 2′,3′,5′,6-tetrafluoro-5-methyl-4′-pentyl-1,2,3,4-tetrahydro-[1,1′-biphenyl]-2-carboxylate

(NMR data derived from subtraction of the pure trans diastereomer)

TLC: R_{f}=0.70 (hexanes/ethyl acetate 9:1). ¹⁹F NMR (CDCl₃, 376 MHz): δ −142.16 (dd, J=21.5, 12.2 Hz, 2F), −146.63 (dd, 2F, J=21.9, 12.3 Hz). ¹H NMR (CDCl₃, 400 MHz): δ 5.40 (d, J=3.1 Hz, 1H), 4.2 (s, 1H), 3.5 (s, 3H), 3.0-2.9 (m, 1H), 2.7 (t, 2H, J=7.6 Hz), 2.25-2.14 (m, 1H), 2.14-1.99 (m, 2H), 1.94-1.83 (m, 1H), 1.73 (s, 3H), 1.62-1.52 (m, 2H), 1.38-1.27 (m, 4H), 0.89 (t, 3H, J=6.7 Hz). ¹³C{1H} NMR (CDCl₃, 101 MHz): δ 174.0, 146.6-154.8 (m), 144.2-143.5 (m), 136.2, 119.4 (t, J=18.9), 117.0 (t, J=14.8 Hz), 51.4, 43.7, 33.3, 31.3, 28.8, 28.7, 23.5, 22.7, 22.3, 21.4, 13.9. HRMS (EI): exact mass calc. for C₂₀H₂₄F₄O₂: m/z=372.1707, found: 372.1706 [M]⁺.

(28) (trans) Methyl 2′,3′,5′,6′-tetrafluoro-5-methyl-4′-pentyl-1,2,3,4-tetrahydro-[1,1′-biphenyl]- 2-carboxylate

TLC: R_{f}=0.70 (hexanes/ethyl acetate 9:1). ¹⁹F NMR (CDCl₃, 376 MHz): δ −144.6 (dd, 2F, J=21.8, 12.5 Hz), -−146.3 (dd, 2F, J=−22.1, 12.5 Hz). ¹H NMR (CDCl₃, 400 MHz): δ 5.19 (s, 1H), 4.11 (d, 1H, J=10.0 Hz), 3.58 (s, 3H), 2.94-2.86 (m, 1H), 2.67 (t, 2H, J=7.6 Hz), 2.24-2.11 (m, 2H), 2.10-2.00 (m, 1H), 1.92-1.79 (m, 1H), 1.70 (s, 3H), 1.63-1.52 (m, 2H+2H impurities), 1.36-1.28 (m, 4H+2H impurities), 0.89 (t, 3H+1H impurities, J=6.87 Hz). ¹³C{¹H}-NMR (CDCl₃, 101 MHz): δ 175.0, 146.3-145.8 (m), 143.8-143.4 (m), 134.6, 120.7, 51.7, 119.4 (t, J=15.7 Hz), 119.0 (t, J=19.0 Hz), 44.2, 34.9, 31.3, 29.3, 28.9, 26.7, 23.2, 22.7, 22.3, 13.9. HRMS (EI): exact mass calc. for C₂₀H₂₄F₄O₂: m/z=372.1707, found: 372.1700 [M]⁺.

(29) 2-(2′,3′,5′,6′-Tetrafluoro-5-methyl-4′-pentyl-1,2,3,4-tetrahydro-[1,1′-biphenyl]-2-yl)propan-2-ol A 3 M solution of MeMgBr in Et₂O (7 equiv.) is added to a 2 M solution of ester (20 mg, 54 μmol) in dry THF while cooling in an ice bath. The solution is warmed to rt and stirred for 30 min. The reaction is quenched with saturated NH₄Cl solution and extracted with ethyl acetate. Column chromatography (20% EA in PE) yields the tertiary alcohol. The product was obtained as a colorless viscous liquid (19 mg, 51 μmol, 95%). cis and trans isomers are separable by column chromatography.

cis-(29)

TLC: R_{f}=0.35 (hexanes/ethyl acetate 9:1). ¹⁹F NMR (CDCl₃, 376 MHz): δ −135.83 -136.11 (m, 1F), 142.71 (dd, 1F, J=22.4, 11.6 Hz), 146.10 (dd, 1F, J=22.4, 12.2 Hz), −146.40 (dd, 1F, J=21.8, 11.7 Hz). ¹H NMR (CDCl₃, 400 MHz): δ 5.30-5.24 (m, 1H), 4.08-4.00 (m, 1H), 2.68 (t, 2H, J=7.6 Hz), 2.19-2.13 (m, 1H), 2.06-1.97 (m, 1H), 1.94-1.76 (m, 2H), 1.70 (s, 3H), 1.63-1.52 (m, 2H), 1.37-1.29 (4H), 1.07 (s, 3H), 1.00 (s, 3H), 0.89 (t, 3H, J=6.9 Hz). ¹³C{¹H} NMR (CDCl₃, 101 MHz): δ 135.7, 120.5, 119.3-118.7 (m), 72.7, 49.5, 32.0, 31.3, 30.8, 28.9, 28.3, 27.2, 23.4, 22.7, 22.3, 20.4, 20.3, 13.9. HRMS (EI): exact mass calc. for C₂₁H₂₆F₄⁺: m/z=354.1965, found: 354.1962 [M−H₂O]⁺.

trans-(29)

TLC: R_{f}=0.40 (hexanes/ethyl acetate 9:1). ¹⁹F NMR (CDCl₃, 376 MHz): −144.51-−144.94 (broad s, 2F), −146.84 (dd, 2F, J=22.02, 12.18 Hz). ¹H NMR (CDCl₃, 400 MHz): δ 5.05 (s, 1H), 3.82 (d, 1H, J=9.3 Hz), 2.65 (t, 2H, J=7.7 Hz), 2.25-2.10 (m, 2 H), 2.03-1.92 (m, 2H), 1.68 (s, 3H), 1.63-1.52 (m, 2H+1H impurities), 1.49-1.38 (m, 1H), 1.36-1.28 (m, 4 H), 1.20 (s, 3H), 1.12 (s, 3H), 0.93 (s, 1H), 0.89 (t, 3H, J=7.0 Hz). ¹³C{¹H} NMR (CDCl₃, 101 MHz): δ 146.3-145.5 (m), 143.9-143.1 (m), 135.2, 123.4 (t, J=15.20 Hz), 117.8 (t, J=18.9 Hz), 122.1, 73.7, 48.0, 34.1, 31.4, 30.2, 29.0, 28.9, 23.2, 22.6, 22.3.

(30) 1,2,4-Trifluoro-6,6,9-trimethyl-3-pentyl-6a,7,8,10a-tetra hydro-6H benzo [c]chromene(1-deoxy-1,2,4-trifluoro-THC)

Procedure derived from Carreira and coworkers.⁷¹ From tertiary alcohol (29) (14 mg, cis:trans=4:1). The tertiary alcohol is dissolved in dry THF. A 1 M solution of KHMDS in THF (1.5 equiv.) is added. The solution is heated to 40° C. in an oil bath for 15 minutes. The reaction is quenched with saturated NH₄Cl solution and extracted with ethyl acetate. Column chromatography (2% EA in PE) yields the target compound as a colorless, viscous liquid. Isolated yield: 13 mg, 37 μmol, cis:trans=4:1, 98%.

cis-(30) ¹⁹F NMR (CDCl₃, 376 MHz): δ −142.75-−142.90 (m, 1F), −145.54 (d, 1F, J=12 Hz), −153.50 (d, 1F, J=22.41 Hz). ¹H NMR (CDCl₃, 400 MHz): δ 6.39-6.31 (m, 1H), 4.03-3.96 (m, 1H), 2.97 (t, J=7.5 Hz, 2H), alkyl region could not be analyzed due to overlaps from both diastereomers. Colorless viscous liquid. HRMS (EI): exact mass calc. for: C₂₁H₂₇F₃O⁺ m/z=352.2009, found: 352.1999 [M]⁺.

trans-(30) ¹⁹F NMR (CDCl₃, 376 MHz): δ −145.04-−145.17 (m, 1F), −145.40 (d, 1F, J=14 Hz), −154.26 (d, 1F, J=22.8 Hz). ¹H NMR (CDCl₃, 400 MHz): δ 6.41 (s, 1H), 3.69 (d, J=11.4 Hz, 1H), alkyl region could not be analyzed due to overlaps from both diastereomers. HRMS (EI): exact mass calc. for: C₂₁H₂₇F₃O⁺ m/z=352.2009, found: 352.2004 [M]⁺.

### Example 2

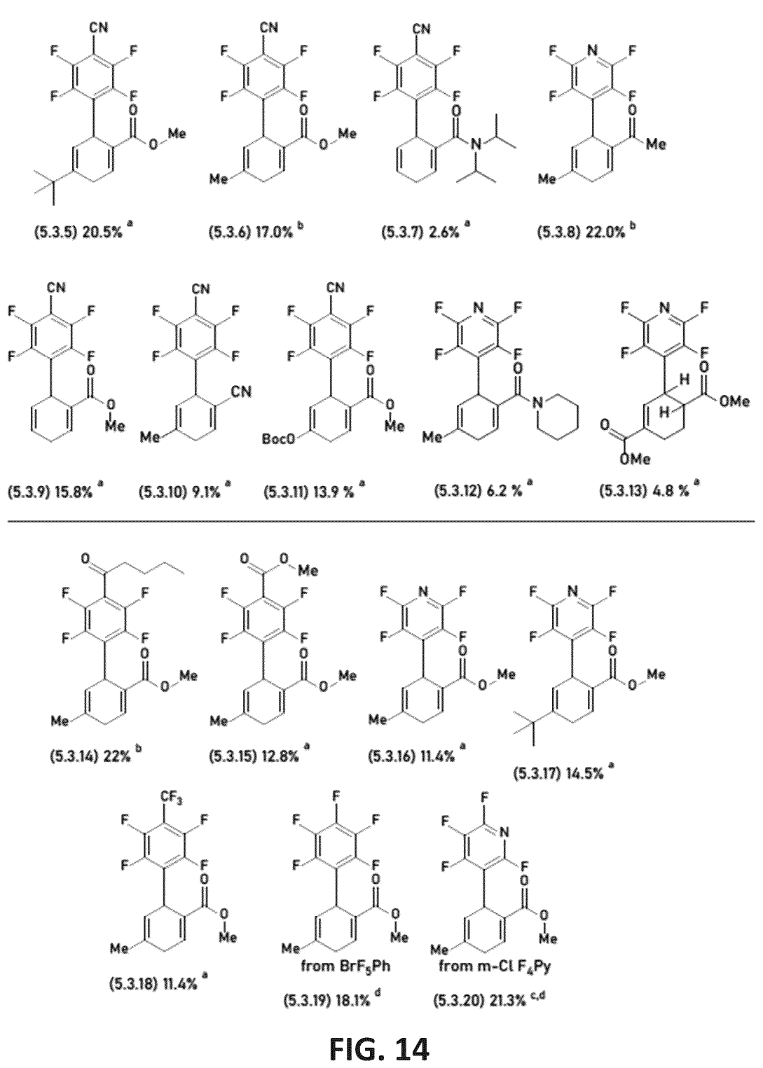

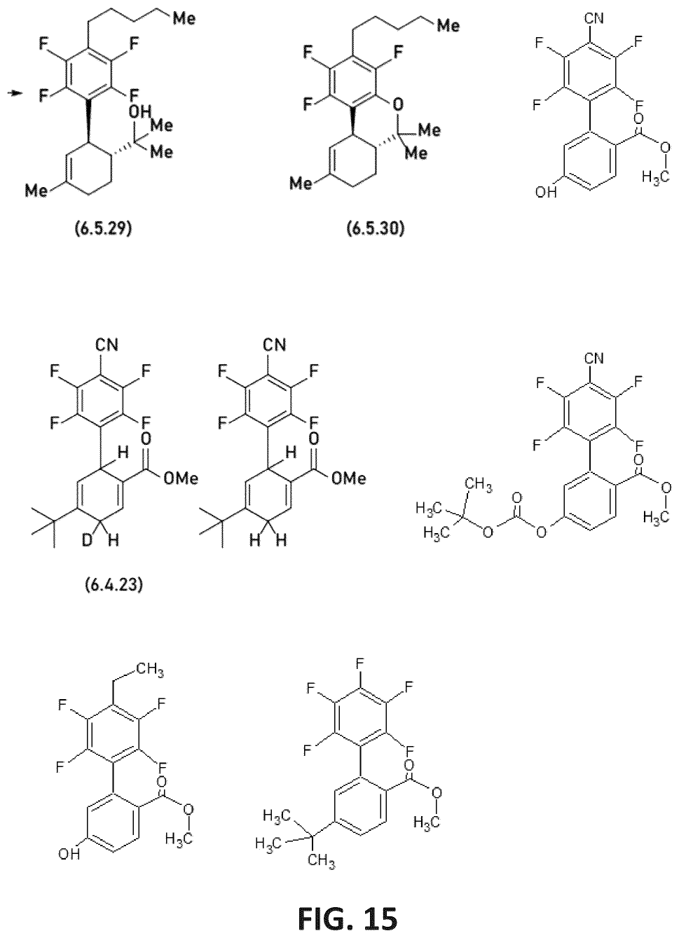

FIGS. 14-15 illustrate the structures of additional non-limiting embodiments of polyfluorinated THC analogues prepared in accordance with the present disclosure.

Thus, in accordance with the present disclosure, there have been provided compounds, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

### REFERENCES

The following references, to the extent that they provide exemplary procedural or other details supplementary to those set forth herein, are specifically incorporated herein by reference. In addition, the following is not intended to be an Information Disclosure Statement; rather, an Information Disclosure Statement in accordance with the provisions of 37 CFR § 1.97 will be submitted separately.
(1) Jaivel et al., J. Nat. Prod. 2014, 77, 2.
(2) Aldemir et al., J. Nat. Prod. 2014, 77, 2331.
(3) Ayoup et al., J. Nat. Prod. 2014, 77, 1249.
(4) Jaivel et al., J. Nat. Prod. 2015, 78, 343.
(5) Murphy et al., Chemosphere 2003, 52, 455.
(6) Walker et al., Chem. Soc. Rev. 2014, 43, 6527.
(7) Fried et al., J. Am. Chem. Soc. 1957, 79, 1130.
(8) Ramsay et al., Clinical Pharmacology & Therapeutics 1976, 20, 167.
(9) Böhm et al., ChemBioChem 2004, 5, 637.
(10) Gillis et al., J. Med. Chem. 2015, 58, 8315.
(11) O'Hagan, D., Chem. Soc. Rev. 2008, 37, 308.
(12) O'Hagan, D., Journal of Fluorine Chemistry 2010, 131, 1071.
(13) Zhan et al., J. Phys. Chem. A 2001, 105, 1296.
(14) Wang et al., Chem Rev 2014, 114, 2432.
(15) Zhou et al., Chem. Sci. 2016, 116, 422.
(16) Mei et al., Chemistry—A European Journal 2019, 25, 11797.
(17) Furuya et al., Synthesis 2010, 2010, 1804.
(18) Sun et al., Org. Lett. 2011, 13, 2750.
(19) Capdevila et al., ACS Catalysis 2019, 9, 11074.
(20) Luo et al., Org. Lett. 2018, 20, 2543.
(21) Senaweera et al., J. Am. Chem. Soc. 2014, 136, 3002.
(22) Chen et al., J. Am. Chem. Soc. 2017, 139, 18444.
(23) Vogt et al., J. Am. Chem. Soc. 2019, 141, 13203.
(24) Matsunami et al., Organometallics 2018, 37, 1958.
(25) Lu et al., J. Am. Chem. Soc. 2016, 138, 15805.
(26) Singh et al., Chem. Sci. 2015, 6, 7206.
(27) Singh et al., Chem. Sci. 2016, 7, 6796.
(28) Senaweera et al., J. Am. Chem. Soc. 2016, 138, 2520.
(29) Nicholls et al., Chem. Comm. 2018, 54, 4589.
(30) Cao et al., Chem. Comm. 2019, 55, 9323.
(31) Dai et al., ACS Catalysis 2018, 8, 802.
(32) Meyer et al., ACS Catalysis 2016, 6, 369.
(33) Bally et al., The Journal of Organic Chemistry 2011, 76, 4818.
(34) Birch, A. J., J. Chem. Soc. (Res.) 1950, 1551.
(35) Chapman et al., J. Am. Chem. Soc. 1963, 85, 41.
(36) McAtee et al., Nature Communications May 2020, 11, 2528.
(37) Dong et al., Org. Lett. 2020, 22, 528.
(38) Gao et al., J. Phys. Chem. A 2009, 113, 6955.
(39) Lowry et al., J. Am. Chem. Soc. 2004, 126, 14129.
(40) Teegardin et al., Org. Process Res. Dev. 2016, 20, 1156.
(41) Daub et al., J. Am. Chem. Soc. 2019, 141, 9543.
(42) Amador et al., Asian Journal of Organic Chemistry 2019, 8, 978.
(43) Chatterjee et al., Angew. Chem., Int. Ed. 2019, 131, 14427.
(44) Singh et al., Chem 2018, 4, 124.
(45) Day et al., J. Am. Chem. Soc. 2018, 140, 9934.
(46) Rathnayake et al., Eur. J. Org. Chem. 2020, 2020, 1433.
(47) Kumarasamy et al., Chem. Rev. 2015, 115, 11239.
(48) Loutfy et al., Canadian Journal of Chemistry 1976, 54, 1454.
(49) Shchegoleva et al., Chem. Phys. Lett. 1999, 312, 325.
(50) Rathnayake et al., Org. Lett. 2019, 21, 9681.
(51) Böhm et al., Chemistry—A European Journal 2016, 22, 15921.
(52) Beatty et al., Acc. Chem. Res. 2015, 48, 1474.
(53) Wayner et al., Chem. Phys. Lett. 1986, 131, 189.
(54) Costentin et al., PNAS 2019, 116, 11147.
(55) Huynh et al., Chem. Rev. 2007, 107, 5004.
(56) Montanari et al., J. Org. Chem. 1993, 58, 5628.
(57) Zhan et al., J. Phys. Chem. A 2004, 108, 2020.
(58) Kelly et al., J. Phys. Chem. B 2006, 110, 16066.
(59) Flamigni et al., In Photochemistry and Photophysics of Coordination Compounds II; Balzani, V., Campagna, S., Eds.; Springer Berlin Heidelberg: Berlin, Heidelberg, 2007, p 143.
(60) Campagna et al., Top. Curr. Chem. 2007, 280, 117.
(61) McTiernan et al., Catalysis Science & Technology 2016, 6, 201.
(62) Bertran et al., J. Am. Chem. Soc. 1996, 118, 5737.
(63) Smothers et al., J. Am. Chem. Soc. 1979, 101, 1895.
(64) Abbas et al., Organic Letters 2018, 20, 868.
(65) Banister et al., ACS Chemical Neuroscience 2019, 10, 2160.
(66) Martin et al., Eur. J. Pharma. 2002, 435, 35.
(67) Westphal et al., J. Am. Chem. Soc. 2017, 139, 18206.
(68) Breuer et al., PLOS ONE 2016, 11, e0158779.
(69) Banister et al., ACS Chemical Neuroscience 2015, 6, 1445.
(70) Barton et al., J. Chem. Soc., Perkin Transactions 1 1975, 1574.
(71) Rackl et al., Beilstein Journal of Organic Chemistry 2014, 10, 2157.
(72) Lau et al., J. Org. Chem. 1986, 51, 3038.
(73) Chandrasekhar et al., Org. & Biomol. Chem. 2006, 4, 1650.

What is claimed is:

1. A compound comprising the structure of Formula I: 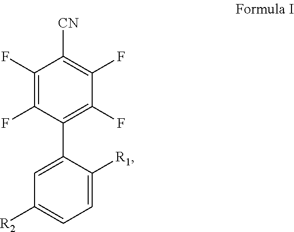
wherein R₁ is a hydroxy, ester, amide, ketone, cyanide, or piperidine group;
wherein R₂ is H, a hydrocarbon, hydroxy, carbonyl, or butyl carbonate group; and
with the proviso that when R₁ is methyl acetate, R₂ is not H or CH₃.

2. The compound of claim 1, wherein R₂ is a hydrocarbon selected from the group consisting of a methyl, ethyl, isopropyl, propyl, tert-butyl, pentyl, prenyl, and iso-prenyl group.

3. The compound of claim 1, wherein R₁ is methyl acetate.

4. The compound of claim 3, wherein R₂ is selected from the group consisting of a hydroxy, methyl, ethyl, isopropyl, propyl, tert-butyl, pentyl, prenyl, iso-prenyl, carbonyl, and butyl carbonyl group.

5. The compound of claim 1, wherein R₁ is CN, and R₂ is CH₃.

6. The compound of claim 1, wherein R₁ is N,N-di(propan-2-yl)acetamide, and R₂ is H or a hydrocarbon.

7. A compound comprising the structure of Formula II: 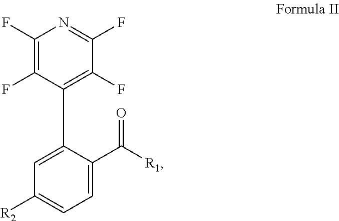
wherein R₁ is a hydroxy, ester, amine, amide, ketone, cyanide, or piperidine group;
wherein R₂ is a hydrocarbon, hydroxy, carbonyl, or butyl carbonate group; and
with the proviso that when R₁ is CH₃ or OCH₃, R₂ is not F or CH₃.

8. The compound of claim 7, wherein R₁ is OCH₃ or methylpiperidine, and R₂ is a hydrocarbon selected from the group consisting of a methyl, ethyl, isopropyl, propyl, tert-butyl, pentyl, prenyl, and iso-prenyl group.

9. The compound of claim 7, wherein R₁ is OCH₃, and R₂ is methyl acetate or a tert-butyl group.

10. The compound of claim 7, wherein R₁ is methylpiperidine, and R₂ is CH₃.

11. A compound comprising the structure of Formula III: 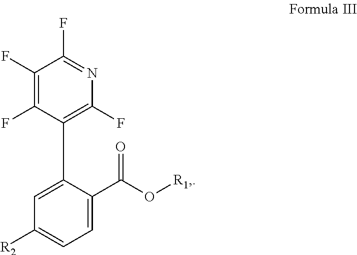
wherein R₁ is a hydroxy, ester, amide, ketone, cyanide, or piperidine group;
wherein R₂ is H, a hydrocarbon, hydroxy, carbonyl, or butyl carbonate group; and
with the proviso that when R₁ is CH₃, R₂ is not H.

12. The compound of claim 11, wherein R₁ is methyl acetate, and R₂ is CH₃.

13. A compound comprising the structure of Formula IV: 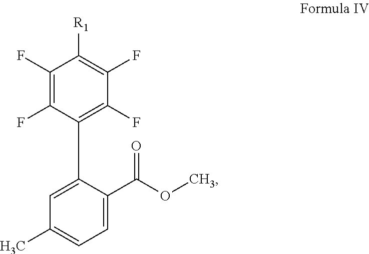
wherein R₁ is H, F, CF₃, a hydroxy, halogen, hydrocarbon, carbonyl, ester, amide, ketone, piperidine, butyl carbonate, or benzoate group; and
with the proviso that R₁ is not CN, a hexanyl group, or a hexan-2-one group.

14. The compound of claim 13, wherein R₁ is an ethyl, methyl acetate, or 2-hexanol group.

15. The compound of claim 13, wherein R₁ is a hexan-2-yl 3,5-bis(trifluoromethyl)benzoate group.

16. A compound comprising the structure of Formula V:

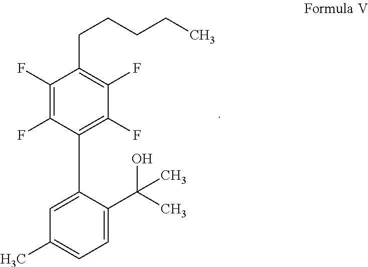 [SMILES: CCCCCc1c(F)c(F)c(-c2cc(C)ccc2C(C)(C)O)c(F)c1F]

17. A compound comprising the structure of Formula VI:

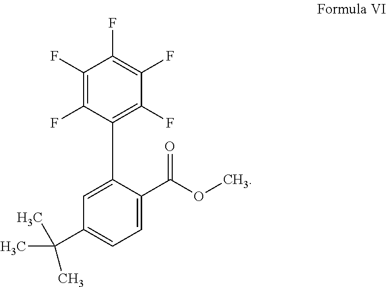 [SMILES: COC(=O)c1ccc(C(C)(C)C)cc1-c1c(F)c(F)c(F)c(F)c1F]

18. A compound comprising the structure of one of the stereoisomers of Formulas VII, VIII, and IX:

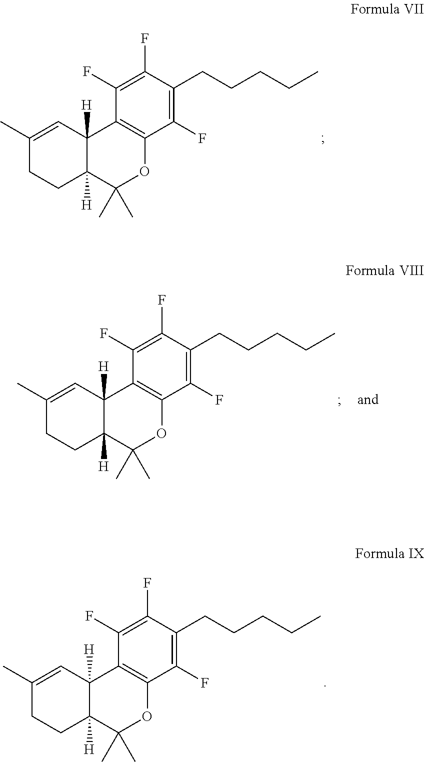

19. A compound comprising the structure of Formula X: 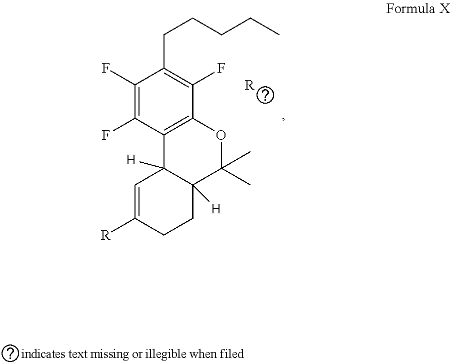
wherein R is H, a hydrocarbon, hydroxy, carbonyl, or butyl carbonate group.

20. A compound comprising the structure of Formula XI: 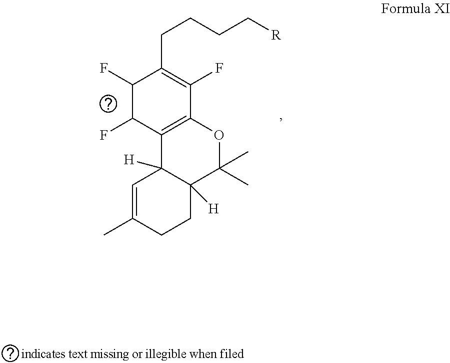
wherein R is a hydrocarbon.

21. The compound of claim 20, wherein R is a C1-C3 hydrocarbon.

22. The compound of claim 20, wherein R comprises an aromatic group.

23. The compound of claim 20, wherein R comprises a phenyl group.

24. The compound of claim 20, wherein R comprises a pentyl group.

25. The compound of claim 20, wherein R is an alkane.

26. A compound comprising the structure of Formula XII: 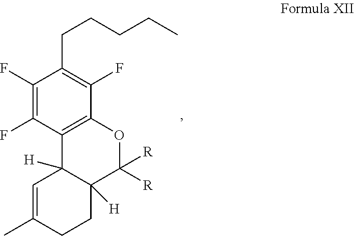
wherein each R is independently selected and is a hydrocarbon.

27. The compound of claim 26, wherein each R is an alkane.

28. A compound comprising the structure of Formula XIII: 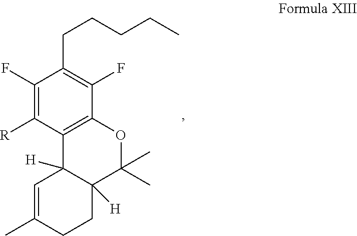
wherein R is F, OH, OR′, SH, SR′, NH₂, or NHR′.

29. The compound of claim 28, wherein R is F, and wherein the compound is a specific stereoisomer thereof.

30. A compound comprising the structure of Formula XIV: 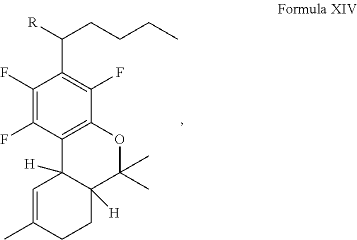
wherein R is a hydrocarbon.

31. The compound of claim 30, wherein R is an alkane.

32. A composition, comprising:
two or more of any of the compounds of claims 1-31.

33. A method, comprising the steps of:
combining a polyfluorinated aromatic compound with an aromatic compound having at least one activated group to form a mixture;
reacting the mixture under conditions that result in coupling with concomitant dearomatization to form a polyfluorinated compound having at least a dicyclic core structure.

34. The method of claim 33, wherein the polyfluorinated compound is a cannabinoid derivative.

35. The method of claim 33, wherein the polyfluorinated aromatic compound has a structure of the following formula: 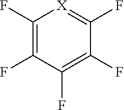
wherein X is N or CR, wherein R is an electron withdrawing group.

36. The method of claim 35, wherein R is selected from the group consisting of a nitrile group, a trifluoromethyl group, a pentanal group, a methyl group, a methyl ester group, a pentyl group, a carbonyl group, a sulfone group, and an alkyne group.

37. The method of claim 33, wherein the polyfluorinated aromatic compound has a structure of the following formula:

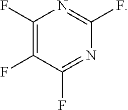

38. The method of claim 33, wherein the polyfluorinated aromatic compound has a structure of the following formula: 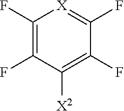
wherein:
X is N, CF, or CR, wherein R is an electron withdrawing group; and
X² is Cl or Br.

39. The method of claim 33, wherein the polyfluorinated aromatic compound is 1-(perfluorophenyl)pentan-1-one.

40. The method of claim 33, wherein the polyfluorinated aromatic compound comprises 1-(trifluoromethyl)-2,3,5,6-tetrafluorobenzene.

41. The method of claim 33, wherein the aromatic compound with at least one activated group has a structure of the following formula: 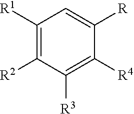
wherein:
R is an electron withdrawing group; and
each of R¹, R², R³, and R⁴ is independently selected from H, a hydrocarbon group having between 1-20 carbons, an aromatic group, and O with a suitable protecting group.

42. The method of claim 41, wherein R is a methyl ester group, each of R¹, R³, and R⁴ is H.

43. The method of claim 42, wherein R² is selected from the group consisting of H, O, OH, CH₃, a tert-butyl group, a methyl ester group, or a BocO group.

44. The method of claim 33, further comprising the step of adding water to the mixture.

45. The method of claim 33, wherein the method produces the compound of any one of claims 1-31.
